(19) United States
(12) Patent Application Publication
(10) Pub. No.: US 2016/0228670 A1
(43) Pub. Date: Aug. 11, 2016

(54) SYSTEM FOR NITRIC OXIDE INHALATION

(71) Applicant: ADVANCED INHALATION THERAPIES (AIT) LTD., Beer-Yaacov (IL)

(72) Inventors: Yossef Av-Gay, Vancouver (CA); David Greenberg, Omer (IL); Racheli Vizman, Beer-Yaacov (IL); Einav Levi, Beit-SheAn (IL)

(21) Appl. No.: 15/021,527

(22) PCT Filed: Sep. 11, 2014
(86) PCT No.: PCT/IL14/50811

§ 371 Date: Mar. 11, 2016

Related U.S. Application Data
(60) Provisional application No. 61/876,346, filed on Sep. 11, 2013.

(51) Int. Cl. A61M 16/12; A61M 16/20; A61M 16/00

(52) U.S. Cl. CPC A61M 16/12; A61M 16/0057; A61M 16/20; A61M 2202/0275; A61M 2205/502

(57) ABSTRACT

A system for inhalation, comprising gas supply apparatus configured to separately supply at least NO, and a carrier gas mixture which contains O₂; a mixer apparatus configured for receiving gases from the supply apparatus and mixing the NO with the carrier gas mixture to provide a therapeutic mixture; an inhaler device configured for receiving the therapeutic mixture and releasing the therapeutic mixture in an enclosed space of the inhaler device; a chemical sensing assembly configured for providing data pertaining to a concentration of at least NO₂ in the inhaler device; and a controller configured for controlling flow of the therapeutic mixture responsively to the data.

### FIELD AND BACKGROUND OF THE INVENTION

The present invention, in some embodiments thereof, relates to a medical system and, more particularly, but not exclusively, to a system for administrating nitric oxide by inhalation.

Nitric oxide (NO) is known to exert highly beneficial pharmacologic effect when inhaled as a gas. Gaseous nitric oxide (gNO) has been found to exert or stimulate antimicrobial and antiviral effect when inhaled at relatively high doses, e.g., higher than 80 ppm.

The reactivity of NO makes its delivery in oxygenating environment complicated, particularly at high concentration which is intended for inhalation. One of the adverse effects of exposing NO to the oxygen in air is the formation of higher oxides of nitrogen that can form by reaction of O₂ with NO, e.g. NO₂, which are potentially harmful to living organisms and tissues.

The effectiveness of NO inhalation treatment also depends on the ability to provide and maintain a certain concentration of NO in the inhaled gas, and apply a certain administration regimen consistently and accurately.

U.S. Pat. Nos. 5,485,827 and 5,873,359 teach devices and methods for treating or preventing bronchoconstriction or reversible pulmonary vasoconstriction in a mammal, effected by causing the mammal to inhale a therapeutically-effective concentration of nitric oxide in a gaseous form or a therapeutically-effective amount of a nitric oxide releasing compound, and an inhaler device containing nitric oxide gas and/or a nitric oxide-releasing compound.

International Patent Application Publication No. WO 2012/114235 teaches a device and a method for generating an intermittent stream of oxygen and nitric oxide mixture, while attempting to reduce the danger of toxic compounds that form in the generated gas stream and while providing therapeutic applications independent from the breathing cycle of a patient.

U.S. Patent Application Publication No. 2004/0129270 teaches devices and methods for administering medical gases.

U.S. Patent Application Publication No. 2010/0051025 teaches systems, compositions and methods for preventing or reducing vasoconstriction in a mammal, involving administering to a mammal a composition containing an artificial oxygen carrier in combination with one or more of a nitric oxide-releasing compound, a therapeutic gas containing nitric oxide, a phosphodiesterase inhibitor, and/or a soluble guanylate cyclase sensitizer.

Additional prior art documents include U.S. Pat. Nos. 7,025,869 and 8,221,800; and International Patent Application Publication Nos. WO 2013/132503, WO 2013/132497, WO 2013/132498, WO 2013/132499 and WO 2013/132500.

### SUMMARY OF THE INVENTION

According to an aspect of some embodiments of the present invention, there is provided a system for inhalation, comprising:

gas supply apparatus configured to separately supply at least NO, and a carrier gas mixture which contains O₂;

a mixer apparatus configured for receiving gases from the supply apparatus and mixing the NO with the carrier gas mixture to provide a therapeutic mixture;

an inhaler device configured for receiving the therapeutic mixture and releasing the therapeutic mixture in an enclosed space of the inhaler device;

a chemical sensing assembly configured for providing data pertaining to a concentration of at least NO₂ in the inhaler device; and

a controller configured for controlling flow of the therapeutic mixture responsively to the data.

In some of any of the embodiments described herein, the chemical sensing assembly is further configured for providing data pertaining to a concentration of each of NO and O₂ independently in the inhaler device.

In some of any of the embodiments described herein, the mixer apparatus comprises:

a mixing chamber formed with a first inlet port for receiving NO gas, and a second inlet port for receiving an additional gas; and

a rotatable member mounted in the mixing chamber and configured for rotating within the mixing chamber so as to mix the NO with the additional gas.

In some of any of the embodiments described herein, the controller comprises:

a data processor configured for receiving the data and calculating flow parameters responsively to the data, the controller being configured for controlling flow of the therapeutic mixture based on the calculated flow parameters.

In some of any of the embodiments described herein, the system presented herein further comprises a graphical user interface (GUI).

In some embodiments, the GUI comprises:

a first display area for displaying a plurality of treatment protocols and allowing a user to select one treatment protocol;

a second display area for displaying controller interface configured to communicate user selection data to the controller;

a third display area for displaying the data during delivery of the therapeutic mixture; and

a fourth display area for displaying treatment log data.

In some of any of the embodiments described herein, the gas supply apparatus comprises a gas reservoir monitoring system.

In some of any of the embodiments described herein, the gas reservoir monitoring system comprises:

a container capable of containing a predetermined amount of pressurized nitric oxide gas (NO);

a movable piston in the container; and

a pressure sensor,

the controller being configured to adjust a position of the piston responsively to pressure data received from the pressure sensor so as to maintain a generally constant pressure level in the container.

In some of any of the embodiments described herein, the gas reservoir monitoring system further comprises a piston position sensor, and the controller being configured to received position data from the piston position sensor, and analyze and display the position data.

In some of any of the embodiments described herein, the gas reservoir monitoring system is connectable to the gas supply apparatus.

In some of any of the embodiments described herein, the inhaler device is a facial respiratory mask or a nasal respiratory mask.

In some of any of the embodiments described herein, the inhaler device is a head respiratory hood.

In some of any of the embodiments described herein, the inhaler device is a whole body respiratory encapsulation.

In some of any of the embodiments described herein, the inhaler device is a respiratory tent or a generally closed enclosure.

According to an aspect of some embodiments of the present invention, there is provided a mixer apparatus for an inhalation system, the mixer apparatus comprising:

a mixing chamber formed with a first inlet port for receiving NO gas, and a second inlet port for receiving an additional gas; and

a rotatable member mounted in the mixing chamber and configured for rotating within the mixing chamber so as to mix the NO with the additional gas to provide a flow of a therapeutic mixture.

According to an aspect of some embodiments of the present invention, there is provided a gas reservoir monitoring system for an inhalation system, comprising:

a container capable of containing a predetermined amount of pressurized nitric oxide gas (NO);

a movable piston in the container;

a pressure sensor;

a piston position sensor; and

a controller configured to adjust a position of the piston responsively to pressure data received from the pressure sensor and to position data received from the piston position sensor, such as to maintain a generally constant pressure level in the container, and analyze the position data,

the gas reservoir monitoring system being connectable to a gas supply apparatus of the inhalation system configured to provide a flow of a therapeutic mixture.

According to an aspect of some embodiments of the present invention, there is provided a graphical user interface (GUI) for an inhalation system having a controller configured for delivering to an inhaler device a therapeutic mixture which comprises NO and for controlling a flow of the therapeutic mixture responsively to a concentration of at least NO₂ in the therapeutic mixture, the GUI comprising:

a first display area for displaying a plurality of treatment protocols and allowing a user to select one treatment protocol;

a controller interface configured to communicate user selection data to the controller; and

a second display area displaying data pertaining to the concentration of the NO₂ in the therapeutic mixture during the delivery of the therapeutic mixture.

According to an aspect of some embodiments of the present invention, there is provided a controller system for an inhalation system, which comprises:

a data processor, configured for receiving data pertaining to concentration of each of NO, O₂ and NO₂ independently and calculating flow parameters responsively to the data; and

a controller configured for controlling flow of a therapeutic mixture which includes NO in the inhalation system based on the calculated flow parameters.

In some embodiments, the controller system is configured for controlling flow of NO responsively to the data pertaining to concentration of NO so as to reach an NO concentration of at least 160 ppm.

In some embodiments, the controller system is configured for actuating an actuatable flushing valve responsively to the data pertaining to concentration of NO₂.

According to an aspect of some embodiments of the present invention, there is provided a system for inhalation, which comprises:

a head respiratory hood adapted to be worn over the head of a subject and having an inlet port and an outlet port; and

a supply and control system configured to introduce into the inlet port a therapeutic mixture which includes NO, to provide data pertaining to a concentration of at least NO₂ in the therapeutic mixture, and to control a flow of the therapeutic mixture responsively to the data.

According to an aspect of some embodiments of the present invention, there is provided a system for inhalation, which comprises:

a whole body respiratory encapsulation adapted to encapsulate the entire body of a subject and having an inlet port and an outlet port; and

a supply and control system configured to introduce into the inlet port a therapeutic mixture which includes NO, to provide data pertaining to a concentration of at least NO₂ in the therapeutic mixture, and to control a flow of the therapeutic mixture responsively to the data.

According to an aspect of some embodiments of the present invention, there is provided a system for inhalation, which comprises:

a generally closed enclosure adapted to contain a plurality of mammalian subjects and having an inlet port and an outlet port; and

a supply and controller system configured to introduce into the inlet port a therapeutic mixture which includes NO, to provide data pertaining to a concentration of at least NO₂ in the therapeutic mixture, and to control a flow of the therapeutic mixture responsively to the data.

In some embodiments pertaining to any one of the systems or apparatus or interface presented herein, the system further comprises an actuatable valve configured responsively to the data pertaining to a concentration of NO₂.

In some embodiments pertaining to any one of the systems or apparatus or interface presented herein, the flow of the therapeutic mixture is synchronized with a breathing of a subject.

In some embodiments pertaining to any one of the systems or apparatus or interface presented herein, the system further includes an actuatable bellows.

In some embodiments pertaining to any one of the systems or apparatus or interface presented herein, the therapeutic mixture includes NO at a concentration of at least 160 ppm.

In some embodiments pertaining to any one of the systems or apparatus or interface presented herein, the therapeutic mixture further includes O₂ at a concentration that ranges from about 20% to about 99%.

In some embodiments pertaining to any one of the systems or apparatus or interface presented herein, the therapeutic mixture may includes NO₂ at a maximal concentration lower than 5 ppm.

Any combination of the embodiments described herein for any one of the systems, apparatus and interface, and respective combinations of these embodiments, is contemplated.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of an exemplary NO inhalation system, according to some embodiments of the present invention;

FIGS. 2A-C are schematic illustrations of exemplary NO mixer apparatus, according to some embodiments of the present invention;

FIG. 3 is a schematic illustration of an exemplary chemical sensing assembly, according to some embodiments of the present invention;

FIGS. 4A-B are schematic illustrations of an exemplary inhaler device which is fitted with an actuatable flushing valve, according to some embodiments of the present invention;

FIG. 5 is a schematic illustration of an exemplary NO inhalation system which includes an actuatable bellows, according to some embodiments of the present invention;

FIGS. 6A-B are schematic illustrations showing an exemplary procedure for initializing a NO inhalation system, according to some embodiments of the present invention;

FIGS. 7A-E are schematic illustrations showing an exemplary procedure for initializing and operating an exemplary NO inhalation system, according to some embodiments of the present invention;

FIG. 8 is a schematic illustration of an exemplary NO mixer combined with an inhaler device in the form of a facial inhalation mask, according to some embodiments of the present invention;

FIGS. 9A-B are schematic illustrations showing an exemplary inhaler device for administering a NO inhalation treatment to a subject placed in a whole body encapsulation, according to some embodiments of the present invention;

FIGS. 10A-B are schematic illustrations showing an exemplary inhaler device for administering a NO inhalation treatment to a subject placed in a head encapsulation, according to some embodiments of the present invention;

FIGS. 11A-B are schematic illustrations showing an exemplary inhaler device for administering a NO inhalation treatment simultaneously to a group of subjects in an inhalation tent/room, according to some embodiments of the present invention;

FIGS. 12A-C are schematic illustrations of exemplary gas reservoir monitoring apparatus, according to some embodiments of the present invention;

FIG. 13 is a flow chart of a method suitable for initializing an exemplary NO inhalation system according to embodiments of the present invention;

FIG. 14 is a flow chart of a method suitable for operating an exemplary NO inhalation system according to embodiments of the present invention;

FIG. 15 is a flow chart of a method suitable for operating an exemplary NO inhalation system according to embodiments of the present invention, which combines the initialization method presented in FIG. 13 and the delivery method presented in FIG. 14; and

FIGS. 16A-D present exemplary graphical elements of an exemplary GUI according to embodiments of the present invention, suitable for operating the NO inhalation system presented herein.

### DESCRIPTION OF SPECIFIC EMBODIMENTS OF THE INVENTION

The present invention, in some embodiments thereof, relates to a medical system and, more particularly, but not exclusively, to a system for administrating nitric oxide by inhalation.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present inventors have devised inhalation systems, and various components thereof, each of which being suitable for delivering (by inhalation) a relatively high concentration of NO to a subject in a safe, accurate and reproducible manner. The NO inhalation systems provided herewith are suitable, for example, and without limitation, for administering NO by inhalation according to an administration regimen as described in International Patent Application Publication Nos. WO 2013/132503, WO 2013/132497, WO 2013/132498, WO 2013/132499 and WO 2013/132500, each of which being incorporated by reference as if fully set forth herein.

While NO is involved in many biological processes and can be harnessed to effect a variety of beneficial pharmacological effects, it is highly reactive and can lead to the formation of deleterious species if not used controllably and efficiently. NO can react spontaneously with ambient oxygen to afford harmful higher oxides, such as nitric dioxide (NO₂). The time required for half NO to be oxidized to NO₂ depends on the concentration in the air, as shown in Table 1 below.

TABLE 1

| NO concentration | t_{1/2} of NO to |
|---|---|
| in air (ppm) | NO2 (minutes) |
| 20,000 | 0.175 |
| 10,000 | 0.35 |
| 1,000 | 3.5 |
| 100 | 35 |
| 10 | 350 |
| 1 | 3500 |

The present embodiments provide a system configured for providing an inhalant comprising NO and oxygen, particularly, but not necessarily in cases in which the concentration of NO is relatively high, e.g., such as but not limited to more than 80 ppm and up to 160 ppm, or higher, and the output of such the inhalant is sufficiently high to allow normal, unassisted and spontaneous breathing of a subject.

Hereinthroughout, whenever the term “nitric oxide” or its abbreviation “NO” is used in the context of inhalation, it is to be understood that nitric oxide is inhaled in the gaseous state. This term is therefore equivalent to, and is used interchangeably with, the terms “gaseous NO”, “gaseous nitric oxide” and “gNO”.

Inhalation System:

In general, administration of nitric oxide gas is accomplished according to some embodiments of the present invention by a designated system that can include a first container of compressed nitric oxide gas in N₂, a second container of oxygen or an oxygen and N₂ mixture and optionally a third container of compressed air or nitrogen, attached to a mixer device that forms a homogenous gas mixture using all or some of the different gas sources. This gas mixture can then pass for inhalation by the subject via an inhaler device, e.g., a mask, a hood, a tent and the like. Controlling the flow of gas from each source and maintaining a designated gas composition, can be achieved according to some embodiments of the present invention by a controller device which can comprise, or be associated with a data processor, such as a general purpose computer or dedicated circuitry. The controller device can optionally and preferably comprise graphical user interface (GUI), for allowing the user to set operational parameters to be used by the controller device. Hence, the system optionally and preferably produces a gas mixture having a predetermined concentration of nitric oxide which is inhaled by the subject and can be maintained at a predetermined NO level for a predetermined period of time repeatedly, consistently and/or accurately.

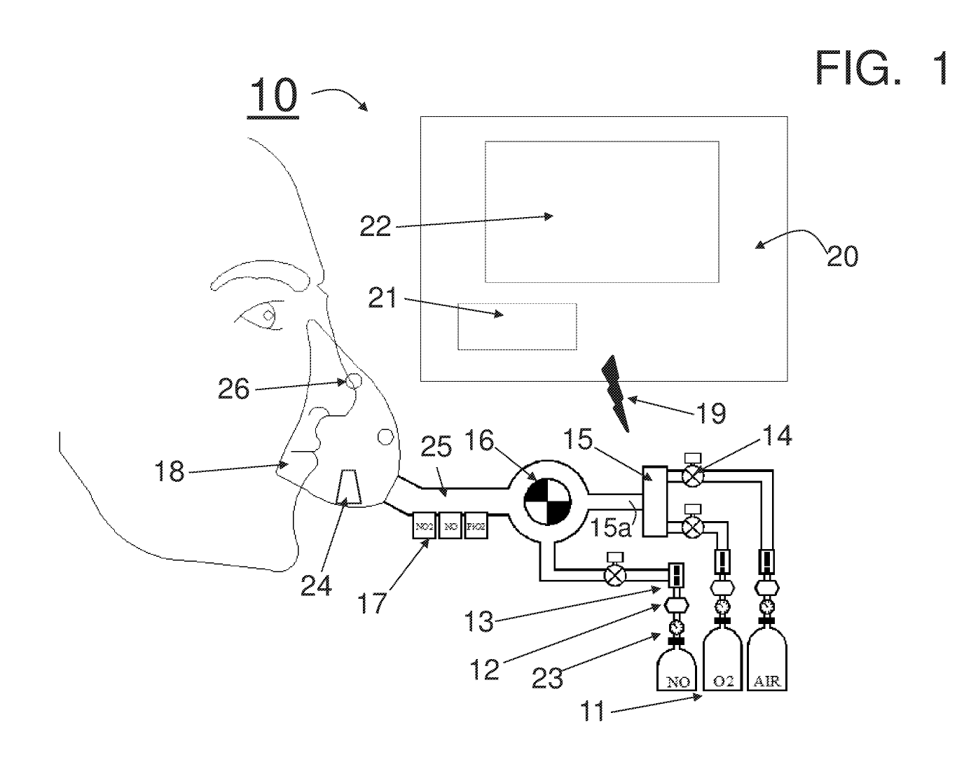

Referring now to the drawings, FIG. 1 illustrates an exemplary NO inhalation system 10, according to some embodiments of the present invention. System 10 can include a plurality of gas source containers 11, each of which being in fluid communication with a respective pressure sensor 23. In the representative illustration of FIG. 1, three containers and three pressure sensors are shown, wherein a first container contains NO, a second container contains oxygen and the third container contains air. However, it is to be understood that it is not intended to limit the scope of the present invention to a configuration which includes three containers and three pressure sensors, and that the present embodiments contemplate any number of containers and sensors.

In some embodiments of the present invention one or more of containers 11 (e.g., each container) is connected to at least one of a pressure regulator 12, a flow meter 13 and an electric valve 14. In various exemplary embodiments of the invention system 10 comprises an oxygen mixer 15 configured for mixing air and/or nitrogen with oxygen (O₂) to provide a carrier mixture of air with O₂ or N₂ with O₂. A NO mixer 16 receives the carrier mixture from O₂ mixer 15, e.g., via a conduit 15a, and mixes NO with the carrier mixture to provide a therapeutic gas mixture.

As used herein “therapeutic mixture” refers to a gaseous mixture which comprises NO and a carrier gas mixture, wherein the carrier gas mixture comprises at least oxygen.

Hence, according to some embodiments of the present invention, the term “therapeutic mixture” refers to a gaseous mixture of NO, oxygen and air or nitrogen, which is characterized by a predetermined, controlled and consistent concentration of NO, O₂ and NO₂. According to some embodiments, the concentration of NO in the therapeutic mixture deviates from the concentration of at least 160 ppm by less than ±10%; the concentration of NO₂ in the therapeutic mixture is less than 5 ppm or less than 2.5 ppm, and the concentration of O₂ in the therapeutic mixture ranges from 21% to 100%, or from 20% to 99% or from 21% to 50%, or from 21% to 30%.

The therapeutic mixture that forms in mixer 16 passes through a chemical sensing assembly 25 having a plurality of chemical sensors 17 configured for sensing the concentration of a at least one gas component in the mixture. Preferably, sensing assembly 25 comprises an NO₂ chemical sensor configured for sensing the level of NO₂. In some embodiments of the present invention sensing assembly 25 comprises an oxygen chemical sensor configured for sensing the level oxygen and in some embodiments of the present invention sensing assembly 25 comprises a nitric oxide chemical sensor configured for sensing the level of nitric oxide. A more detailed description of sensing assembly 25 is provided hereinunder.

It is stated that regulations of NO and NO₂ requires NO and NO₂ sensors placed in the patient breathing circuit.

From assembly 25, the therapeutic gas mixture flows into an inhaler device 18 having at least one actuatable flushing valve 24 and at least one passive outlet vent 26. Also contemplated, are embodiments in which sensing assembly 25 is part of inhaler device 18 as further detailed hereinbelow. Inhaler device 18 can be provided, for example, in the form of facial respiratory mask, as illustrated in FIG. 1. Other types of inhaler devices include, without limitation, a head encapsulating respiratory device (e.g., a hood) and a whole body encapsulating respiratory device (e.g., a tent). A more detailed description of the principle and operations of valve 24 and vent 26 is provided hereinunder.

In various exemplary embodiments of the invention system 10 includes a controller 20, which is in communication via a wireless or wired communication link 19 with at least one of: pressure sensors 23, pressure regulator 12, flow meter 13, electric valve 14, O₂ mixer 15, NO mixer 16, chemical sensor 17 and actuatable flushing valve 24. Controller 20 further includes a data processor 21 and a graphical user interface (GUI) 22. Data processor 21 can include a central processing unit (CPU) which can be a part of a general purpose computer or dedicated circuitry, and is in communication with GUI 22 via link 19. GUI 22 allows the user to interact with data processor 21 through graphical icons and visual indicators so as to set operational parameters to be used by processor 21 (input) and to receive information therefrom (output).

The NO inhalation system of the present embodiments is configured to deliver a mixture of gases at a flow rate that allow a subject to breath normally at a resting state, namely 1-15 liters per minute. The system of the present embodiments can also be configured to exhibit a low inspiratory resistance. The required peak output of the inhalant of the NO inhalation system of the present embodiments preferably supply at least a flow that exceeds (by about 10-30%) or at least matches the peak expiratory flow (PEF; also referred to as peak expiratory flow rate or PEFR) of a normally breathing healthy adult subject. For example, normal peak expiratory flow rate of adult humans may range from 420 liters per minute (L/min) to 670 L/min, depending on sex, age, height and weight, while pediatric PEFR values may range from 80 L/min to 400 L/min.

According to embodiment of the present invention, the nitric oxide may be provided in any commercially available form such as high pressure cylinders (e.g., P_{g} of about from about 2000 psi to about 2400 psi, e.g., 2200 psi) which contain NO at a concentration of about 800 ppm in an inert carrier gas (e.g., N₂ or Ar). Nitric oxide can also be provided in other forms, such as low pressure disposable cylinders (e.g., P_{g} of from about 100 psi to about 200 psi, e.g., about 150 psi) which contain NO at a concentration of up to about 5000 ppm or more in an inert carrier gas. In the context of any embodiment of the present invention, it is noted that nitric oxide can be generated from N₂ and O₂ (i.e., air) by using an electric nitric oxide generator (as disclosed in, e.g., U.S. Pat. No. 5,396,882 to Zapol), and further noted that NO can also be mixed with room air, using a standard low-flow gas mixer (e.g., Bird Blender, Palm Springs, Calif., USA).

According to embodiments of the present invention, exhaled gases are collected and diverted to an exhaust outlet rather than allowed to be mixed with ambient atmosphere. For example, the exhaled gases can be filtered, e.g. with a HEPA filter, or collected by a gaseous-waste scrubbing device.

The NO inhalation system according to embodiments of the present invention can further comprise tubing, ducts, pumps, valves, seals, bellows, fans and the likes. In various exemplary embodiments of the invention the components of the system are selected suitable for handling a flow of 1-15 liters per minute and are chemically resistant against exposure to NO in a concentration of at least 200 ppm.

The NO inhalation system, according to some embodiments of the present invention, meets the accepted requirements for a home use device in terms of electrical and medical safety specifications. According to some embodiments of the present invention, the system can be operated using a standard AC power source, or a battery as a sole electrical power source, which can be connected to a wall-mount DC power supply charger, and be monitored by a battery capacity indicator.

In general, the NO inhalation system, according to some embodiments of the present invention, meets FDA and CE guidelines, including the design control requirements as described in the FDA's QSR and ISO 13485, and meets the requirements of IEC 60601-1 (2004-11) for medical electrical equipment.

The present embodiments relate to a number of components of a NO inhalation system as disclosed herein, and embodiments of the present invention relate to a design of each of these components. It is also noted that each of the components presented herein can be used within a NO inhalation system in combination with any number of the other components presented herein or all of the components presented herein. It is also noted that each of the components presented herein can be used within a NO inhalation system in combination with any number of alternative components that can be used for the same purpose, e.g., with alternative components which are known at the time of conception of the present invention or will be available in future.

It is noted that the inhalation systems presented herein can be used to provide and deliver any gas mixture. In the context of some embodiments of the present invention, the system presented herein is designed also to produce deliver a therapeutic mixture of gases for inhalation by a subject, which is defined in, e.g. PCT/IL2013/050219.

NO Mixer:

In order to reduce, retard or prevent the formation of higher oxides of nitrogen that form spontaneously upon contact of O₂ with NO, and particularly NO₂, the NO mixer of the present embodiments is designed to introduce the carrier mixture and NO such that a the therapeutic mixture is formed, passed and used rapidly e.g., in less than 2 minutes or less than 1 minute or less than 40 seconds or less than 30 seconds or less than 20 seconds or less than 10 seconds (see, Table 1 hereinabove) and consistently, e.g., with a tolerance of less than 50%, 25% or 10% relative to a minimum value of 5 ppm NO₂, 2.5 ppm, or 1.255 ppm of NO₂ in the inhaled composition of gases.

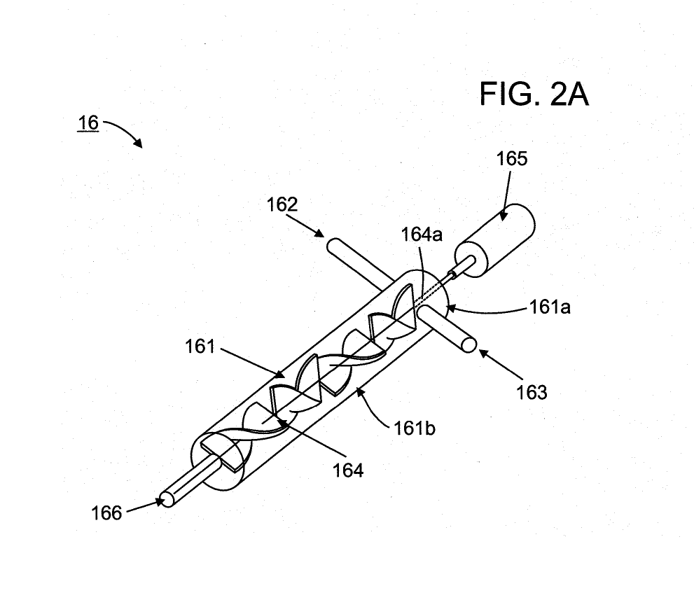

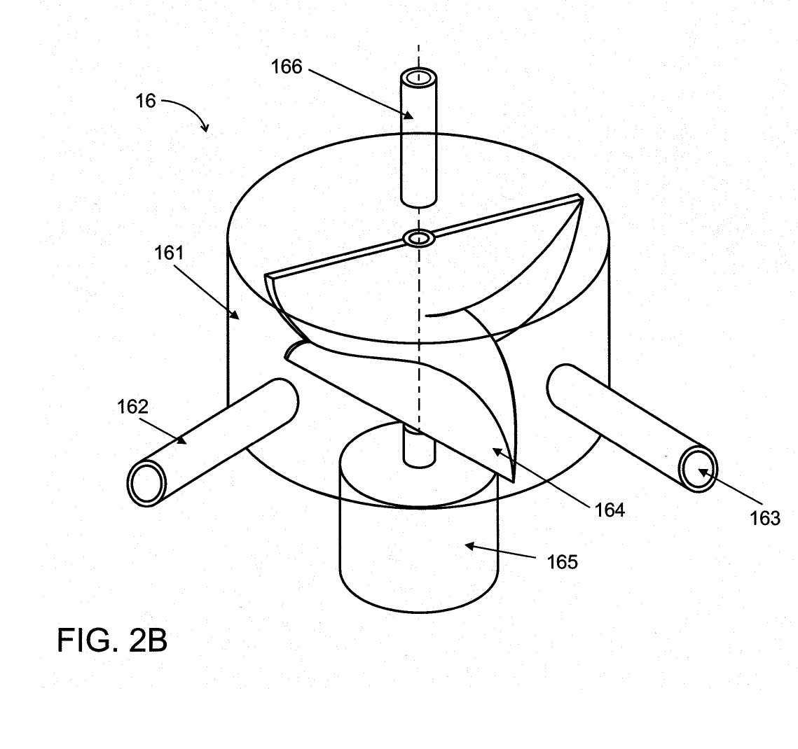

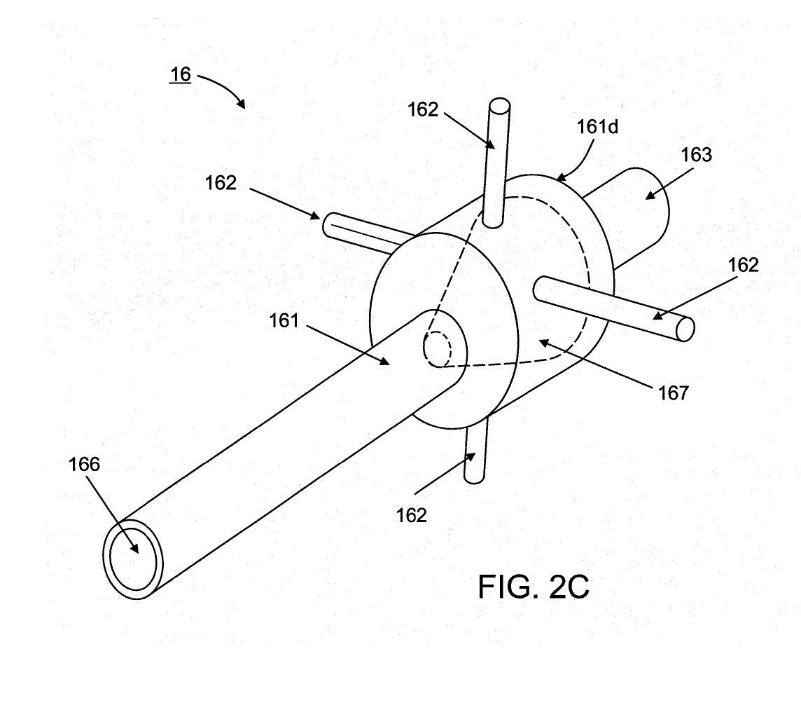

Representative examples of NO mixers, according to some embodiments of the present invention are illustrated in FIGS. 2A-C.

FIG. 2A illustrates an exemplary NO mixer 16, which comprises a mixing chamber 161, carrier mixture inlet 162, a NO inlet 163, a rotating member 164, an electric motor 165 and an outlet 166. In operation, inlets 162 and 163 feed gases into mixing chamber 161. For example, inlet 162 can feed carrier mixture from O₂ mixer 15 (see FIG. 1) and inlet 163 can feed NO from NO container 11 (see FIG. 1). FIG. 2A illustrates an embodiment in which chamber 161 has an elongated tubular shape. Specifically, the diameter to length ratio of chamber 161 is relatively small (e.g., less that 0.5 or less than 0.1 or less than 0.02). However, this need not necessarily be the case, since chamber 161 can have any shape. Further, FIG. 2A illustrates a configuration in which inlets 162 and 163 are generally collinear (e.g., within 10%) with each other at opposite sides of chamber 161. However this need not necessarily be the case, since in some embodiments it is not necessary for inlets 162 and 163 to be generally collinear. In some embodiments, inlets 162 and 163 are positioned such that one inlet is close to end 161a opposite outlet 166 and the other inlet is positioned near center 161b of chamber 161, thereby allowing the gradual introduction of one gas into the other, whereas each of the incoming gases can be let in through any inlet.

Within chamber 161 there is a rotating element 164 having an axis of rotation 164a connected to motor 165. Motor 165 provides rotating element 164 with a rotary motion within chamber 161. In the schematic illustration of FIG. 2A, rotating element 164 is illustrated as a helical member, but other shapes of rotating elements are not excluded from the scope of the present invention, such as a finned rotating element, a plurality of vanes or blades connected to a shaft, and the like.

FIG. 2B illustrates NO mixer 16 in embodiments in which chamber 161 has a larger diameter to length ratio (e.g., at least 1 or at least 2 or at least 3 or at least 4), and inlets 162 and 163 are non-collinear (e.g., at an angle of from about 75° to about 105° to each other.

FIG. 2C illustrates NO mixer 16 in embodiments of the invention in which mixer 16 is devoid of a rotation element. In these embodiments, mixer 16 comprises a conic member 167 within chamber 161, wherein the wide side of member 167 is facing base 161d of chamber 161. Several inlets 162 (four are illustrated in FIG. 2C) are oriented radially and distributed circumferentially along the periphery of chamber 161, and extend inwardly to feed gas (preferably the carrier mixture) into member 167 in the radial direction to establish a turbulent flow within conic member 167. Inlets 163 is preferably mounted at the back side of chamber 161 and arranged to feed the other gas (preferably the NO) along the axial direction into the wide side of member 167.

Chemical Sensing Assembly:

Once the therapeutic mixture is formed, it passes through a chemical sensing assembly in which the mixture is analyzed for its chemical composition with respect to the concentration of at least NO, O₂ and NO₂. To that end, the chemical sensing assembly is fitted with chemical sensors for detecting each of at least NO, O₂ and NO₂ in the therapeutic mixture, by means of at least one NO sensor, at least one FiO₂ sensor and at least one NO₂ sensor, respectively.

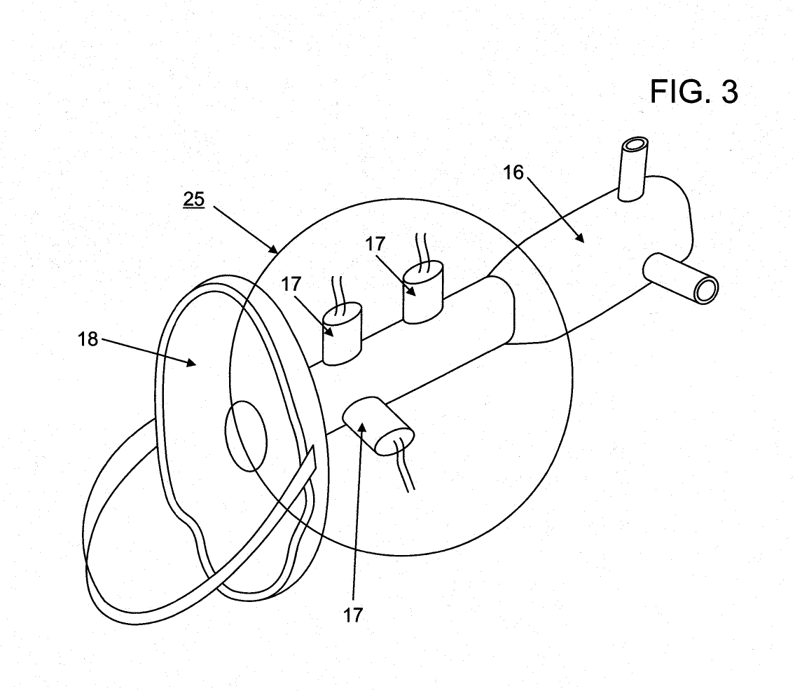

FIG. 3 illustrates chemical sensing assembly 25, which bridges between mixer 16 and inhaler device 18, and which comprises a plurality of chemical sensors 17, each for determining the concentration of one of at least NO, O₂ and NO₂. FIG. 3 is schematic illustration of system 10 in embodiments in which inhaler device 18 is provided in the form of a facial respiratory mask. However, this need not necessarily be the case, since, for some applications, it may not be necessary for device 18 to be a mask. One of ordinary skills in the art, provided with the details described herein would know how to connect and operate assembly 25 for the case of other types of inhaler devices, such as, but not limited to, a respiratory hood or a whole body encapsulation.

Nitric oxide and nitric dioxide detectors suitable for the present embodiments are described in, for example, U.S. Patent Application Publication Nos. 20070181444 and 20100282245, U.S. Pat. Nos. 5,603,820, 7,897,399, 7,914,664 and 8,057,742, and International Patent Application Publication No. WO 2008/088780, the contents of which are hereby incorporated by reference.

Actuatable Flushing Valve:

In various exemplary embodiments of the invention the formation of higher nitrogen oxides, such as nitric dioxide, is prevented during the administration of the therapeutic mixture to a subject. This can be done by monitoring the signal received from the NO₂ chemical sensor. When the concentration of NO₂ in the therapeutic mixture is above a predetermined threshold, the gaseous content of inhaler device is preferably evacuated out of the device. A typical value for the NO₂ threshold is, without limitation, from about 1 ppm to about 10 ppm or from about 1 ppm to about 5 ppm, or from about 1 ppm to about 2.5 ppm e.g., about 2.5 ppm. The gaseous content is preferably evacuated using a valve, such as, but not limited to, actuatable flushing valve 24 (see FIG. 1).

Thus, in operation, data processor 21 receives signals from assembly 25 and analyses these signals to determine the concentration of NO₂ near sensors 17. Data processor 21 compares the NO₂ concentration to the NO₂ threshold. If the NO₂ concentration is above the threshold, controller 20 transmits actuation signal to open actuatable flushing valve 24 so that the gaseous content is evacuated. Optionally and preferably, mixer 16 continues to provide a gaseous mixture, so that an influx fresh therapeutic mixture replaces the content of the inhaler device. The actuatable flushing valve is preferably maintained in its open state until the signals from assembly 25 indicates that the level of NO₂ near sensor 17 is below or not above the threshold.

In some embodiments, the inhaler device comprises one or more passive outlet vent(s) 26 to allow excess therapeutic mixture and exhalation of the subject. In various exemplary embodiments of the invention the opening that forms when actuatable flushing valve 24 opens, in terms of area, is larger (e.g., at least 2 or at least 3 or at least 4 or at least 5 or at least 10 times larger) than the opening of any of passive outlet vent(s) 26 so as to allow rapid replacement of the inner atmosphere of the inhaler device.

According to some embodiments, when the level of NO₂ exceeds the threshold and actuatable flushing valve 24 opens, controller 20 also sends signals to pressure regulators 12 so as to increase the flow of gasses out of containers 11 thereby increasing the influx of the therapeutic mixture into device 18. According to some embodiments, the controller sends signals to pressure regulators 12 so as to reduce, at least temporarily, the pressure of NO that enters mixture 16. This reduces the concentration of NO in the therapeutic mixture and thereby assists in reducing the formation of higher nitric oxides in the inhaled gas mixture. The system resumes the normal production and delivery of the therapeutic mixture once the NO₂ chemical sensor reads the acceptable level thereof.

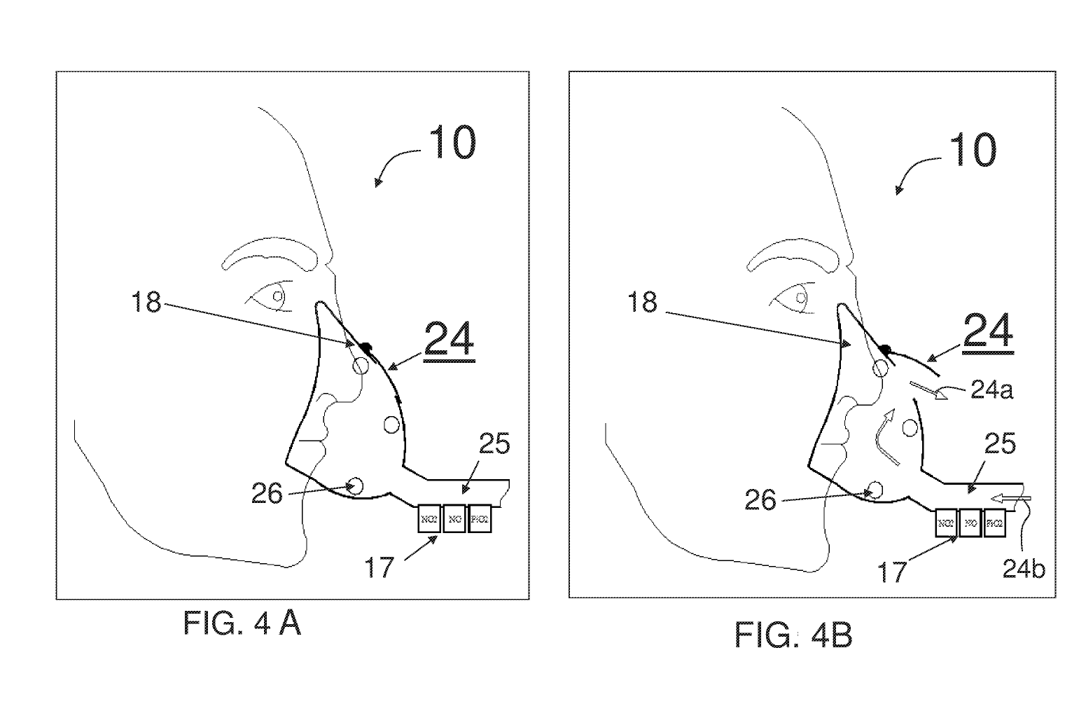

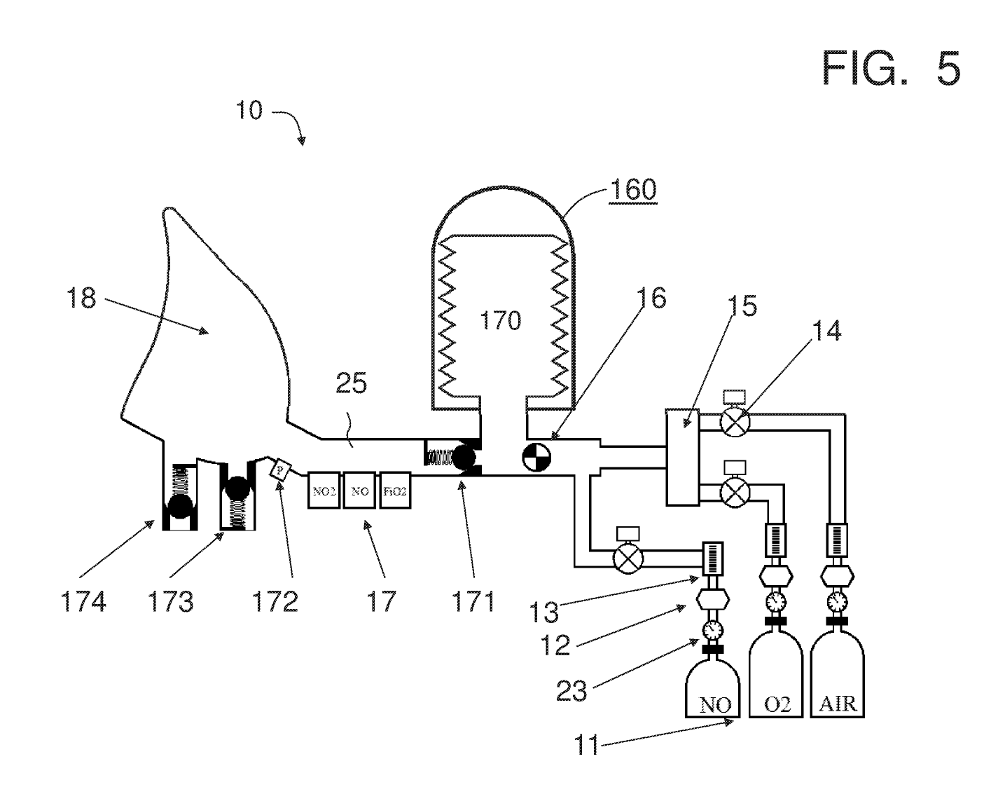

FIGS. 4A-B illustrate an exemplary configuration for inhaler device 18, wherein FIG. 4A shows actuatable flushing valve 24 in a closed state (no efflux through valve 24), and FIG. 4B shows actuatable flushing valve 24 in an open state position, showing efflux 24a of gases out of inhaler device 18 and influx 24b of therapeutic mixture that passes through sensing assembly 25.

Actuatable Bellows:

In some embodiments of the present invention, NO inhalation system 10 comprises an actuatable bellows 160. These embodiments are illustrated in FIG. 5.

Actuatable bellows 160 assists in supplying the therapeutic mixture to inhaler device 18. In some embodiments, system 10 is configured to deliver a therapeutic mixture at an amount which is approximately the same as the amount of gas exhaled by a subject in a single breathing cycle. In these embodiments, pressure changes in inhaler device 18 are monitored. Bellows 160 preferably comprises a contractible bag 170 having a volume selected in accordance with the average lung volume capacity of the subject using the system.

Tables 2A-B present average lung volume and capacity, respectively, of healthy adult humans.

TABLE 2A

| | Value (liters) | |
|---|---|---|
| Volume parameter | In men | In women |
| Inspiratory reserve “sigh” volume or (IRV) | 3.3 | 1.9 |
| Tidal volume (TV) | 0.5 | 0.5 |
| Expiratory reserve volume (ERV) | 1.0 | 0.7 |
| Residual volume (RV) | 1.2 | 1.1 |

TABLE 2B

| | Average value | | |
|---|---|---|---|
| | (liters) | | |
| Volume parameter | In men | In women | Derivation |
| Vital capacity (VC) | 4.8 | 3.1 | IRV plus TV plus ERV |
| Inspiratory capacity (IC) | 3.8 | 2.4 | IRV plus TV |
| Functional residual | 2.2 | 1.8 | ERV plus RV |
| capacity (FRC) | | | |
| Total lung | 6.0 | 4.2 | IRV plus TV plus |
| capacity (TLC) | | | ERV plus RV |

The tidal volume, vital capacity, inspiratory capacity and expiratory reserve volume can be measured directly with a spirometer. Thus, for example, when system 10 is designed for an adult male subject, the volume of bag 170 provides at least the IRV of the subject to provide the maximal volume which occurs during normal breathing (also known as the sigh volume), and can be from about 3.3 to about 3.5 liters.

Actuatable bellows 160 optionally and preferably receives actuation signals from controller 20 (not shown, see FIG. 1) which receives input from an electric one-way valve 171, positioned between chemical sensing assembly 25 and actuatable bellows 160. Valve 171 is optionally responsive to changes in the difference between the pressure at the output side of valve 171 (on the side assembly 25) and the pressure at the input side of valve 172 (on the side of bellows 160). Specifically, when the pressure at the input side is lower than the pressure at the output side, one-way valve 171 opens, and when the pressures are reversed or equal, one-way valve 171 closes.

In various exemplary embodiments of the invention information pertaining to the state of valve 171 is obtained by controller. For example, when valve 171 is open, a signal can be transmitted by valve 171 to controller 20, and when valve 171 is closed the signal to the controller can cease. Controller 20 actuates bellows 160 responsively to the state of valve 171. Specifically, when valve 171 is open, controller 20 signals the actuatable bellows to contract and to deliver the therapeutic mixture into assembly 25, and when valve 171 is close controller 20 returns the actuatable bellows returns to is extended position, thereby pulling fresh therapeutic mixture from NO mixer 16. In was found by the present inventors that operation ensures synchronization between the delivery of the therapeutic mixture and the breathing cycle of the subject, because lower pressure at the output side of valve 171 is correlative with the inhale phase, wherein higher pressure at the output of valve 171 is correlative with the exhale phase.

In some embodiments, the inhaler device comprises an inhaler pressure sensor 172, which can be used to monitor the pressure in device 18 and optionally to activate the electric one-way valve 171. For example, sensor 172 can transmit signals indicative of the pressure in device 18 to controller 20. Based on these signal controller 20 can operate bellows 160 so that the contraction and expansion of bellows 160 is synchronized with pressure variations within device 18, hence also with the breathing cycle of the subject.

System 10 can further comprise at least one of an inhaler outlet valve 173, and an inhaler inlet valve 174. Inhaler outlet valve 173 allows excess therapeutic mixture and exhalation of the subject to exit inhaler device 18, and inhaler inlet valve 174 allows ambient air to enter inhaler device 18, e.g., in case of a system failure.

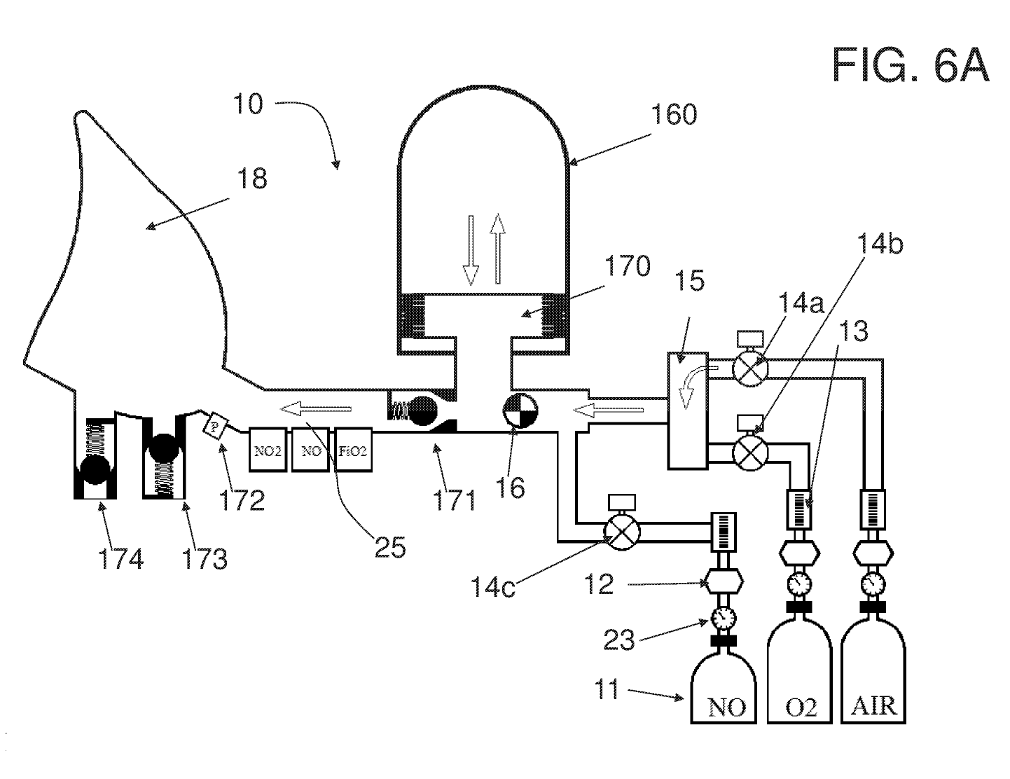

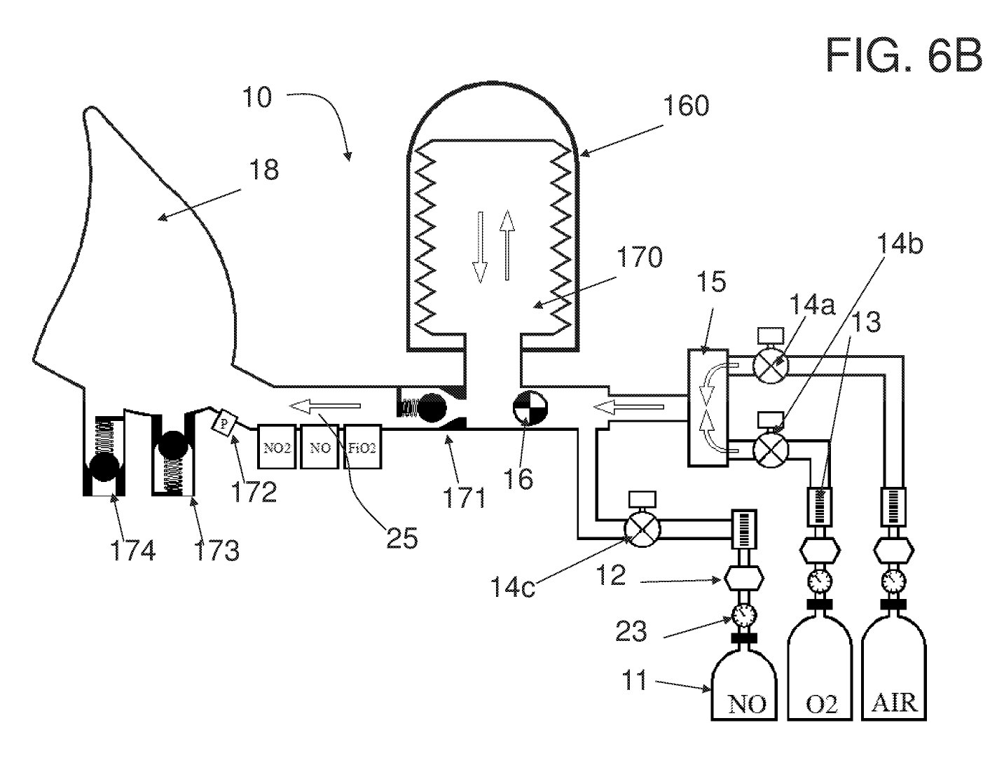

FIGS. 6A-B are schematic illustrations showing a preferred procedure for initializing system 10, according to some embodiments of the present invention. FIGS. 6A-B are schematic illustrations of system 10 in embodiments in which inhaler device 18 is provided in the form of a facial respiratory mask. However, this need not necessarily be the case, since, for some applications, it may not be necessary for device 18 to be a mask. One of ordinary skills in the art, provided with the details described herein would know how to execute the procedure for the case of other types of inhaler devices, such as, but not limited to, a respiratory hood or a respiratory tent.

FIG. 6A illustrates a first initialization stage of system 10 before it is used by a subject. In this stage only valve 14a of the air container is open, while valve 14b of the O₂ container and valve 14c of the NO container are closed. The signals for opening valve 14a and closing valves 14b and 14c can be transmitted by controller 20, responsively to a user command entered, e.g., via interface 22. Bellows 160 is operated for a few (e.g., 2-10) cycles and the entire volume of inhaler device 18 is filled with only with air from the air container.

FIG. 6B illustrates a second initialization stage of system 10 before it is used by a subject. In this stage, valve 14a of the air container and valve 14b of the O₂ container are open and valve 14c of the NO container is closed. The signals for opening valves 14a and 14b and closing valve 14c can be transmitted by controller 20, responsively to a user command entered, e.g., via interface 22. Bellows 160 is operated for a few (e.g., 2-10) cycles and the entire volume of inhaler device 18 is filled with the carrier mixture only, without NO.

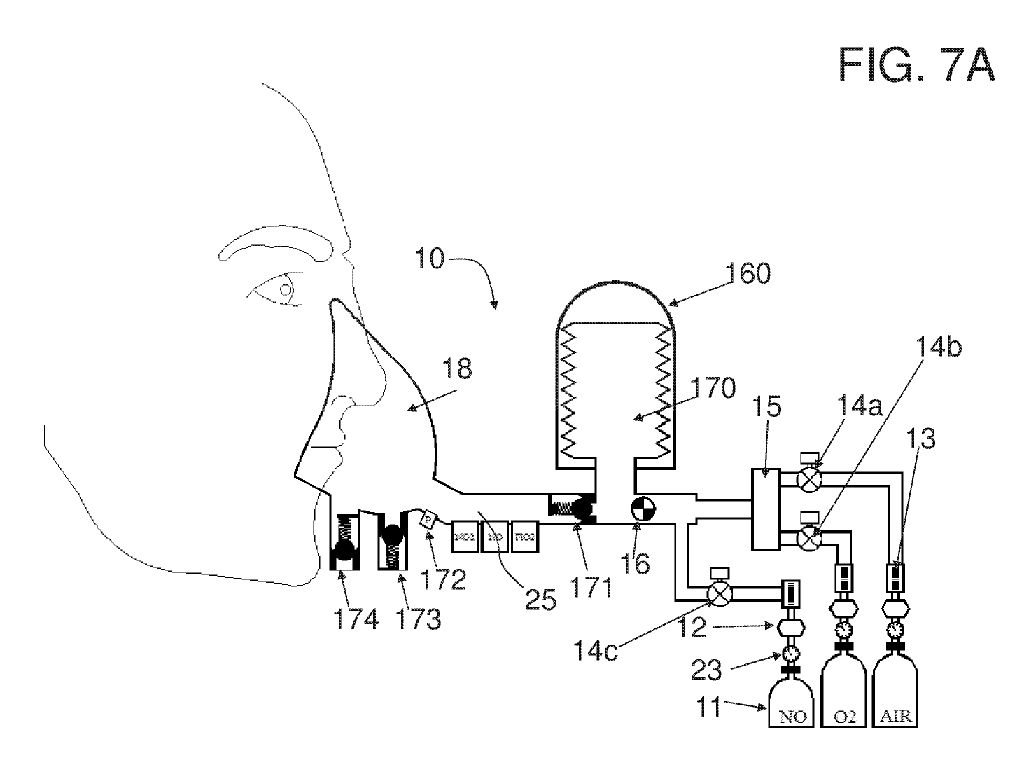

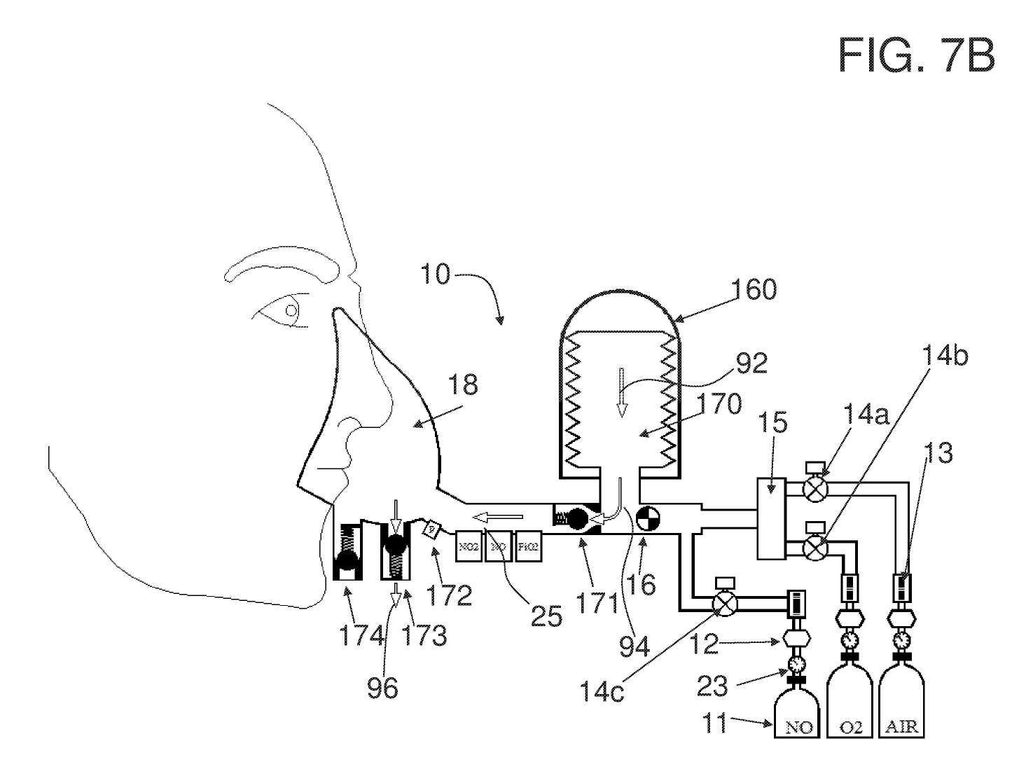

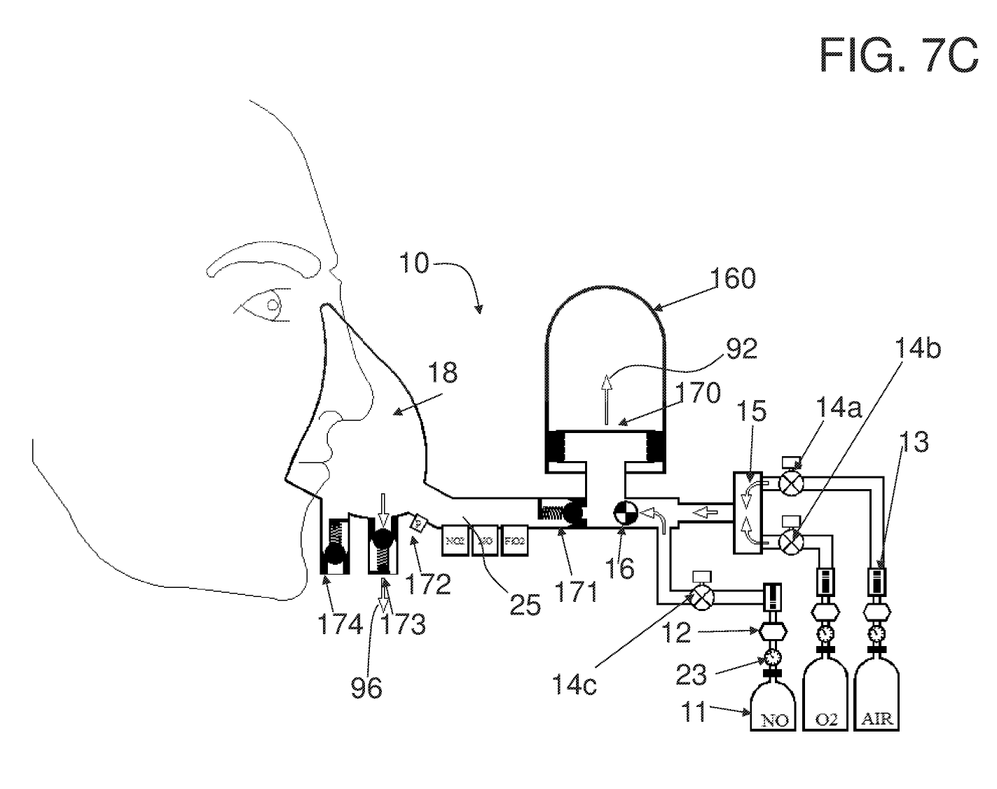

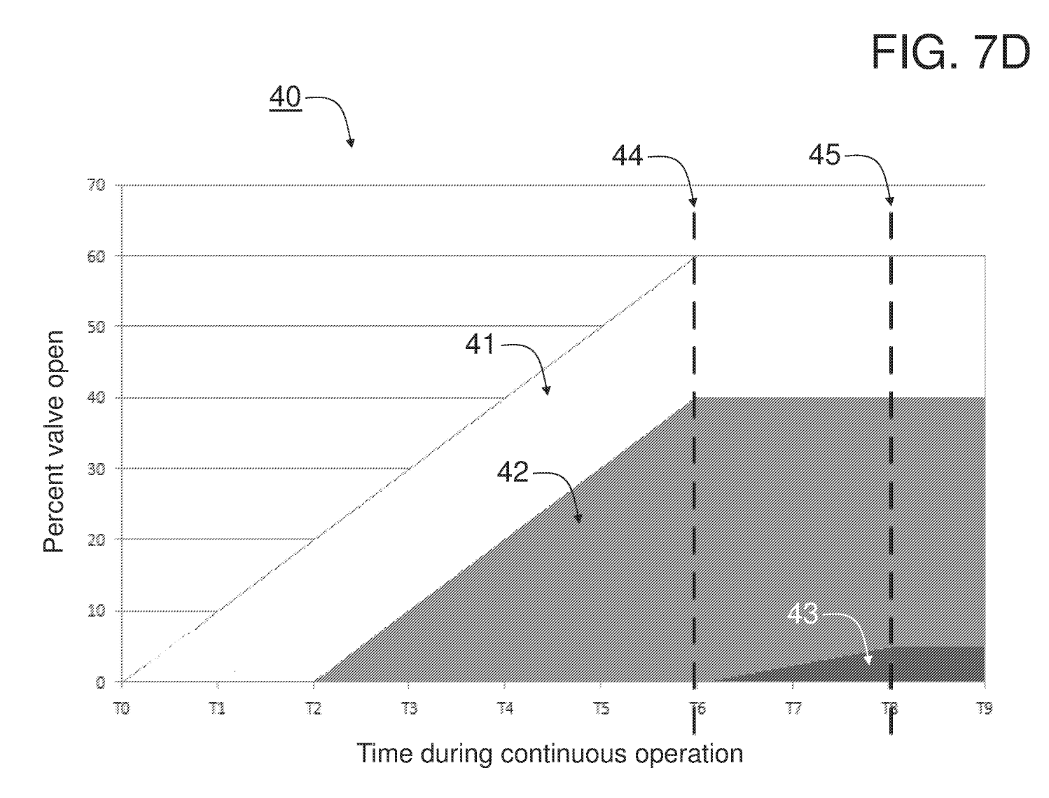

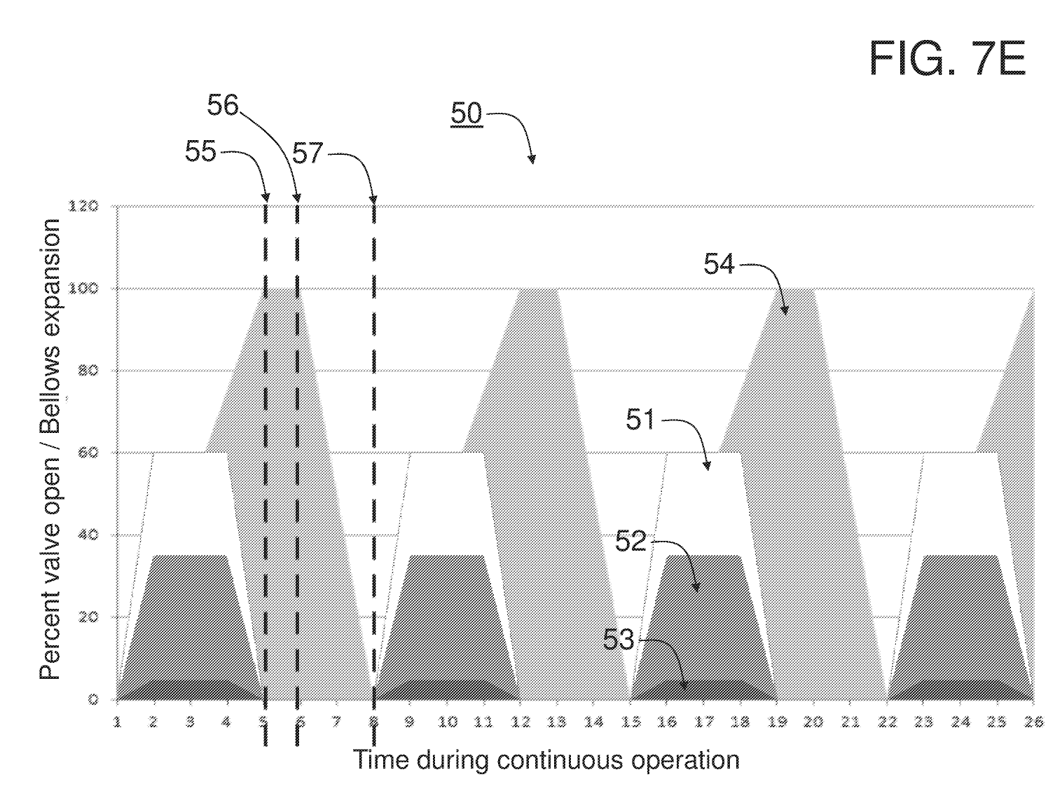

A representative cycle of operation of system 10 is illustrated in FIGS. 7A-E. FIGS. 7A-E are schematic illustrations of system 10 in embodiments in which inhaler device 18 is provided in the form of a facial respiratory mask. However, this need not necessarily be the case, since, for some applications, it may not be necessary for device 18 to be a mask. One of ordinary skills in the art, provided with the details described herein would know how to execute the cycle for the case of other types of inhaler devices, such as, but not limited to, a respiratory hood or a respiratory tent.

FIG. 7A illustrates system 10 in its ready state wherein device 18 and actuatable bellows 160 are filled with therapeutic mixture.

FIG. 7B illustrates system 10 as it responds to the subject's inhale phase which creates an under-pressure in inhaler device 18 so that the pressure is lower at the output side than at the input side of valve 171. Electric one-way valve 171 opens and actuatable bellows 160 contracts 92 to push 94 therapeutic mixture into inhaler device 18 while excess therapeutic mixture exits 96 inhaler device 18 through inhaler outlet valve 173.

FIG. 7C illustrates system 10 as it responds to the subject's exhale phase which creates over-pressure in inhaler device 18 so that the pressure is higher at the output side than at the input side of electric one-way valve 171. Electric one-way valve 171 closes and actuatable bellows 160 expands 92 to take in therapeutic mixture into bag 170, while excess therapeutic mixture and subject's exhalation exits 96 inhaler device 18 through inhaler outlet valve 173.

FIG. 7D presents graph 40 showing the opening of the gas air/N₂ valve 14a and the oxygen valve 14b (expressed in percentage as a function of time on an arbitrary time scale) of during a typical initiation of system 10. Carrier gas valve activity 41 and oxygen valve activity 42 rise up to time point 44 at which the FiO2 reaches a desired level. At time point 44 NO valve activity 43 rises up to time point 45 at which the nitric oxide level reaches the desired level.

FIG. 7E presents graph 50 showing the concerted opening of valves 14a, 14b and 14c and bellows expansion 92 (expressed in percentage) during a typical operation of system 10. Carrier gas valve activity 51, oxygen valve activity 52 and nitric oxide valve activity 53 increase, maintain and decrease in coordination with bellows expansion 54 so as to fill the bellows to bellow full. At time point 55 bellows maintain capacity until subject's inhale phase is detected at time point 56, to which bellows responds by contraction at time point 57.

In some embodiments of the present invention, sensing assembly 25 is provided as part of inhaler device 18. In some embodiments, inhaler device 18 is further configured for mixing gases to provide the therapeutic mixture within the internal space of device 18, as illustrated in FIG. 8.

FIG. 8 illustrates an exemplary inhaler device in the form of a facial inhalation mask, which acts as a NO mixer and a chemical sensing assembly, wherein inhaler device 18 comprises a series of chemical sensors 17 that monitor and send signals to controller 20 via communication links 19 that allow data processor 21 in controller 20 to analyze the input and compute parameters to adjust each of electric valves 14 independently so as to control the input of gases entering the inhaler device via carrier mixture inlet 31 and NO inlet 32, and/or open or close actuatable flushing valve 24 in response to the input from each of chemical sensors 17. Nozzle 33 can be a single nozzle or a plurality of nozzles branching off of inlet 32, and enter inhaler device 18 at various different locations so as to further promote even and homogeneous dispersion of NO in the space enclosed by inhaler device 18. In such embodiments, the relatively small volume of inhaler device 18 allows the carrier mixture to mix with the NO in situ thereby minimize the formation of higher nitrogen oxides, while being monitored by chemical sensors 17.

Data processor 21 receives signals from chemical sensors 17, and uses these signals to provide control data to controller 20. Controller 20 uses the data to operate valves 14a and 14b so as to control the flow of carrier mixture from O₂ mixer 15, and to operate valve 14c which lets NO flow into inhaler device 18 so as to provide a predetermined chemical composition for the therapeutic mixture. Data processor 21 also utilize the signals from sensors 17 to determine the concentration of NO₂ and control the actuatable flushing valve 24 in inhaler device 18. For example, when data processor 21 determines that the threshold for acceptable level of NO₂ is exceeded, controller 20 sends a signal that opens the actuatable flushing valve 24 and keeps it open until the level of NO₂ returns to acceptable values.

In some embodiments, opening the actuatable flushing valve is accompanied with the controller lowering or arresting the flow of NO into the inhaler device by controlling valve 14c, and increasing the flow of carrier mixture via O₂ mixer 15, thereby flushing the content of the inhaler device from the undesired NO₂. This embodiment is advantageous in terms of exposure of NO to O₂, which is kept to a minimum before the therapeutic mixture is inhaled.

Inhaler Device:

The present embodiments are useful for treating any subject including, without limitation, an infant, a small child, a shallow-breathed subject, a single normally breathing subject or a group of any of the above.

It is recognized by the present inventors that each subject or group of subjects exhibits a different breathing pattern and/or a different average individual or collective IRV. For example, an infant typically breathes short shallow breaths with a relatively small IRV compared to an adult subject; a single normally breathing subject has a rhythmic breath, while a group of subjects exhibits a relatively large and uncoordinated cumulative IRV compared to a single subject. In various exemplary embodiments of the invention subjects of different groups are treated with inhaler devices of different types and sizes.

The delivery volume of system 10 is preferably selected in accordance with the expected characteristic IRV of the subject.

The term “delivery volume”, as used herein, refers to the volume containing the therapeutic mixture, which is measured from and including the actuatable bellows (in embodiments in which the bellows is employed), through the chemical sensing assembly up to the distal ends of the inhalation device.

An exemplary inhalation system device having a delivery volume which is about equal to the subject's IRV may be exemplified by a system having an inhaler device in the form of an inhalation mask. An inhalation mask may be used as an inhaler device of an exemplary NO inhalation system as presented herein for any subject that can be fitted with an inhalation mask in terms of face-size and physical ability, and that can breathe normally so as to inhale and exhale through the valves of the system.

In case of an infant or a frail subject or a shallow-breathed subject the inhaler device preferably has a relatively large delivery volume compared to the expected characteristic IRV of the subject so as not to burden the subject's breathing while maintaining a consistent flow of the therapeutic mixture for inhalation by the subject. Typical delivery volume in these embodiments is from about 1 liter to about 10 liters/min.

In some embodiments the delivery volume is about equal to the expected characteristic IRV of the subject. This embodiment is particularly useful when the subject is a normally breathing adult subject that can breathe normally so as to inhale and exhale through the valves of the system. In these embodiments, the inhaler device may be, for example, a facial respiratory mask or a nasal respiratory mask. Typical delivery volume in these embodiments is from about 0.5 liter to about 5 liters.

In some embodiments the delivery volume is larger (e.g., at least 2 times, or at least 3 times, or at least 4 times, or at least 10-times or larger) than the expected characteristic IRV of the subject. This embodiment is particularly useful when the subject is an infant. In these embodiments, the inhaler device comprises, for example, a head encapsulation or whole body encapsulation. Such inhaler devices are useful in any case where the subject is weak, small or unable to have a face mask attached to its face for any reason. Typical delivery volume in these embodiments is from about 20 liters to about 100 liters.

In some embodiments, the delivery volume is at least 10 times larger, or at least 20 times larger, or at least 30-times larger than the expected collective IRV of a plurality of subjects. This embodiment is particularly useful when referring to a group of subjects which is treated simultaneously. In such embodiment, the inhaler device may be, for example, an inhalation tent or an inhalation room. Typical delivery volume in these embodiments is from about 6 cubic meters to about 20 cubic meters.

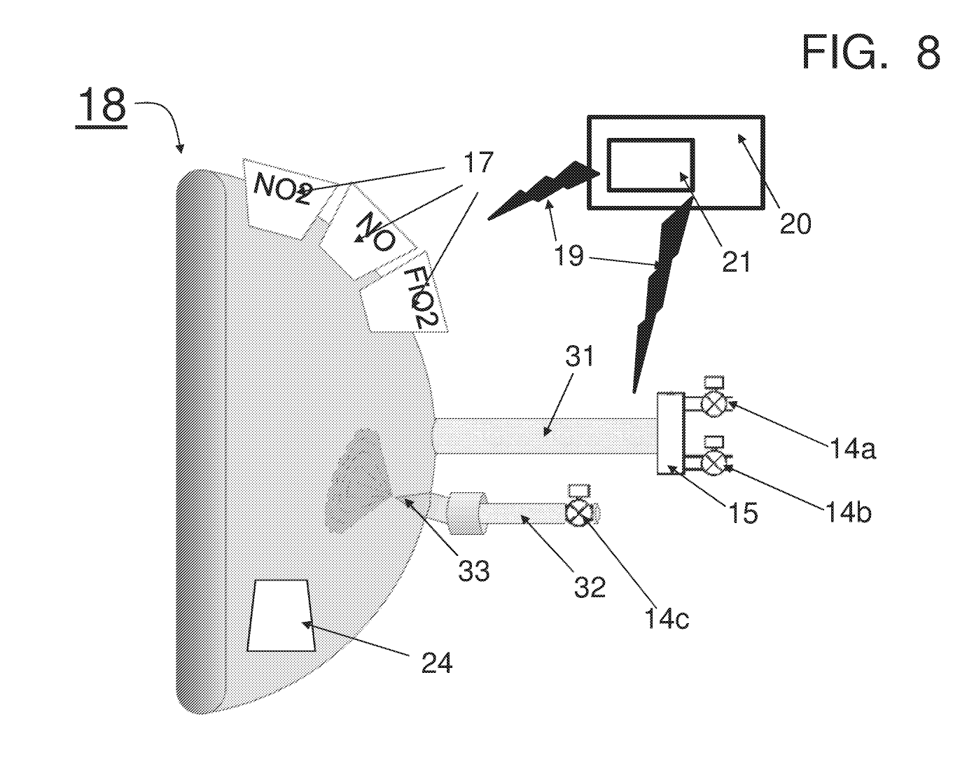

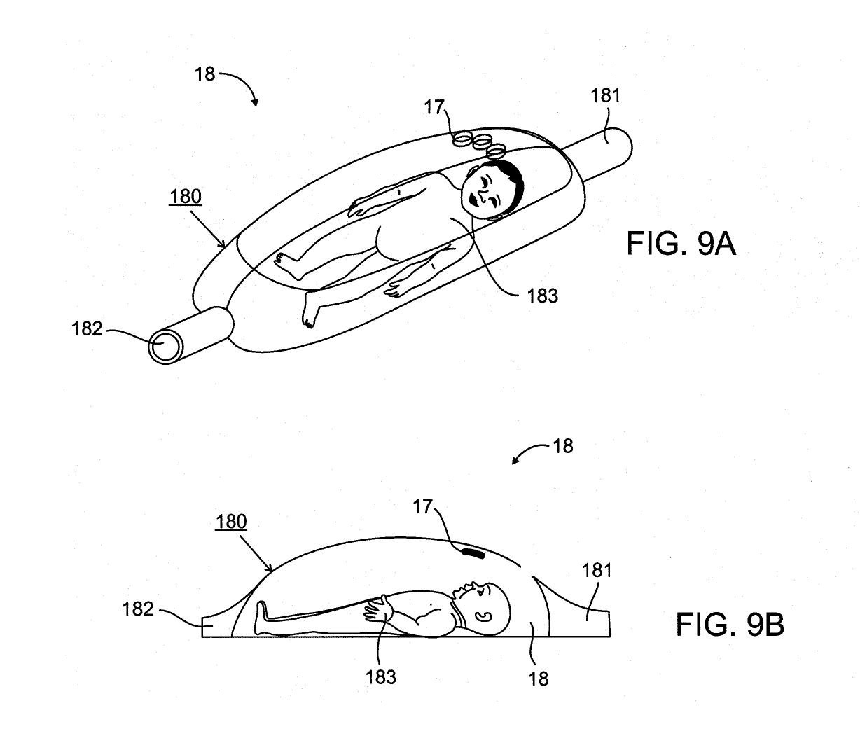

FIGS. 9A-B are schematic illustrations of an isometric view (FIG. 9A) and a side view (FIG. 9B) of inhaler device 18 in embodiments of the invention in which inhaler device 18 is a whole body inhalation device which comprises a whole body encapsulation 180. Whole body encapsulation 180 is useful when the treated subject 183 is an infant, wherein the entire body of the infant is introduced into encapsulation 180. The dimensions of whole body encapsulation are typically from about 40 to about 80 cm in length and from about 30 to about 50 cm in diameter. Embodiments in which whole body encapsulation 180 is sizewise compatible with the dimensions of a child (e.g., from about 80 to about 150 cm in length and from about 40 to about 80 cm in diameter) or an adult (e.g., from about 150 to about 220 cm in length and from about 70 to about 110 cm in diameter) are not excluded from the scope of the present invention.

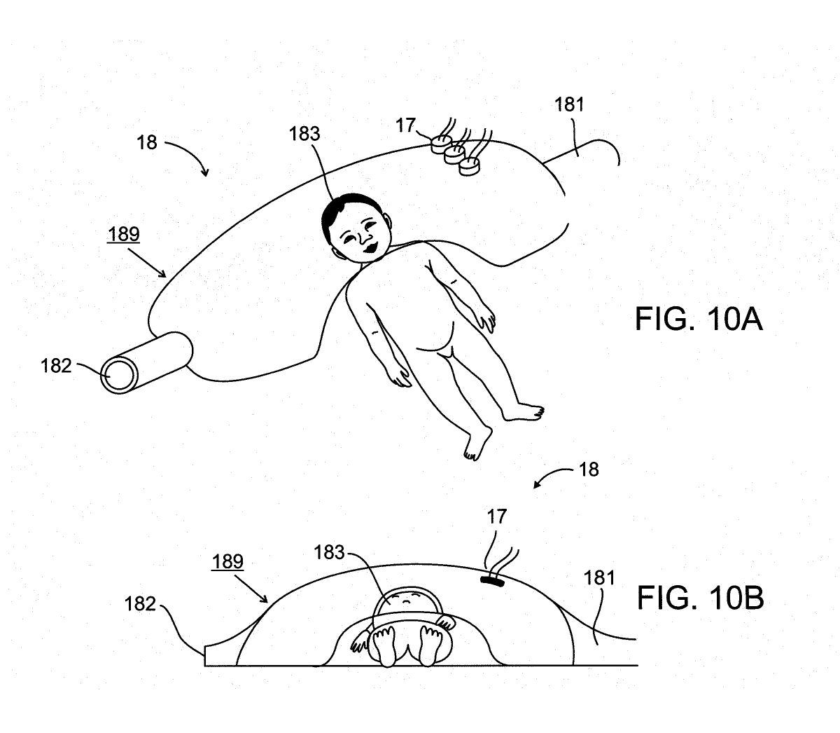

FIGS. 10A-B are schematic illustrations of an isometric view (FIG. 10A) and a side view (FIG. 10B) of inhaler device 18 in embodiments of the invention in which inhaler device 18 is a hood inhalation device which comprises a head encapsulation 189. Head encapsulation 180 is useful when the treated subject 183 is incapable of wearing a facial mask for any reason, in which case the subject's head is introduced into head encapsulation 189. The dimensions of the head encapsulation are typically from about 40 to about 80 cm in length and/or width and from about 30 to about 50 cm in height. Embodiments in which head encapsulation 189 is sizewise compatible with the dimensions of a head of a child (e.g., from about 40 to about 60 cm in length and/or width and from about 30 to about 40 cm in height) or an adult (e.g., from about 50 to about 80 cm in length and/or width and from about 40 to about 50 cm in height) are contemplated. Embodiments in which head encapsulation 189 is sizewise larger than in the examples presented herein, is not excluded from the scope of the present invention.

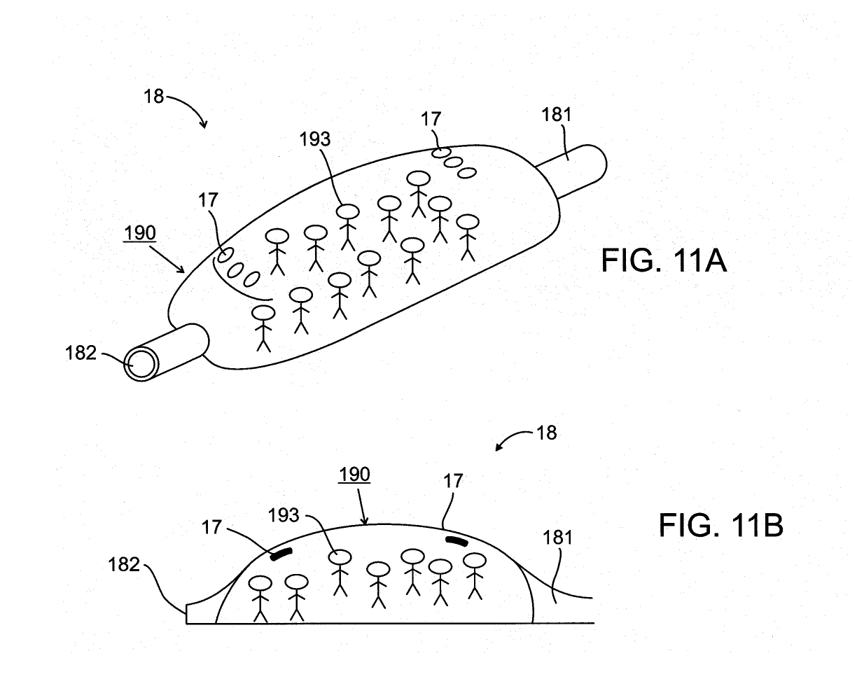

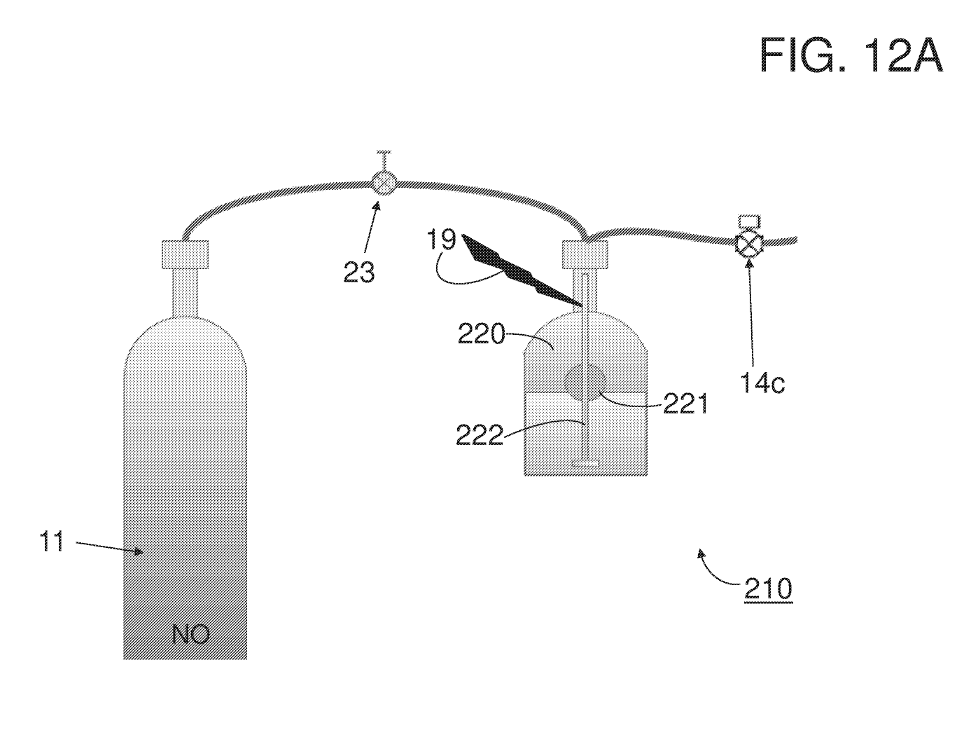

According to embodiments of the present invention, system 10 can be used to collectively treat a group of subjects, wherein the inhaler device is embodied as an inhalation tent or an inhalation room. These embodiments are schematically illustrated in FIGS. 11A-B.

FIGS. 11A-B are schematic illustrations of an isometric view (FIG. 11A) and a side view (FIG. 11B) of inhaler device 18 in embodiments of the invention in which inhaler device 18 is an inhalation tent which comprises group encapsulation 190. Group encapsulation 190 is useful for treating a group of subjects 193 at once, in which case the subjects are introduced into group encapsulation 190. The dimensions of the tent are typically from about 2 meters to about 10 meters in length and/or width and from about 2 meters to about 4 meters in height. Embodiments in which group encapsulation 190 is sizewise compatible with a smaller of a larger group of subjects are not excluded from the scope of the present invention.

Group encapsulation 190 can be with or without a solid construction, wherein for the former the construction can be internal or external, and wherein for the latter the shape of the tent is maintained by the internal gas pressure.

Chemical sensors 17 can be mounted on a wall of whole body encapsulation 180 or head encapsulation 189 or group encapsulation 190, and the therapeutic mixture can be introduced into whole body encapsulation 180 or head encapsulation 189 or group encapsulation 190 through an inlet 181. Inlet 181 can also be connected to sensing assembly, such as assembly 25, which is external to whole body encapsulation 180 or head encapsulation 189 or group encapsulation 190, as illustrated in FIGS. 1-8 for the case of facial inhalation mask. In this embodiment the wall of whole body encapsulation 180 or head encapsulation 189 or group encapsulation 190 can be provided without sensors 17. The ordinarily skilled person, provided with the details described herein would know how to connect inlet 181 of whole body encapsulation 180 or head encapsulation 189 or group encapsulation 190 to assembly 25.

The therapeutic mixture can be provided by containers 11 O₂ mixer 15 and mixer 16 as further detailed hereinabove.

Gases, which typically include excess amount of therapeutic mixture and exhaled gas can be allowed to exit whole body encapsulation 180 or head encapsulation 189 or group encapsulation 190 through an outlet 182. The gasses can be released to the environment or be collected using a gas waste collecting scrubbing device (not shown).

In some exemplary embodiments of system 10, the inhaler device illustrated in FIGS. 11A-B is in a form of an inhalation room, wherein the subject(s) enter or otherwise introduced into the room for any given period of time according to the specified treatment regimen. In such embodiments of the invention care is being take to monitor the atmosphere in group encapsulation 190 at several independent locations, thus the system is fitted with more than one chemical sensing assembly. The system according to the embodiments illustrated in FIGS. 11A-B can comprise suitable tubing, ducts, pumps, valves, bellows, fans and the likes which are suitable to handle volumes of gas lager than 6, 10, 16 or larger than 20 cubic meters. The controller's program of such system is optionally and preferably configured to respond within a sufficiently short time period (e.g., less than 30 seconds or less than 20 seconds or less than 10 seconds) to large gas volumes, in initialization, treatment and cases of NO₂ flushing.

NO Reservoir Monitoring System:

The NO inhalation systems provided herein can comprise a system that inter-alia provides the operator an indication of the amount of NO available for operating the system. Such indication is useful to determine if there is sufficient NO left in the container to complete at least one treatment cycle, as discussed herein. The feature which is capable of indicating the amount of NO left in the system can be embodied as a gas reservoir monitoring system, or GRM system.

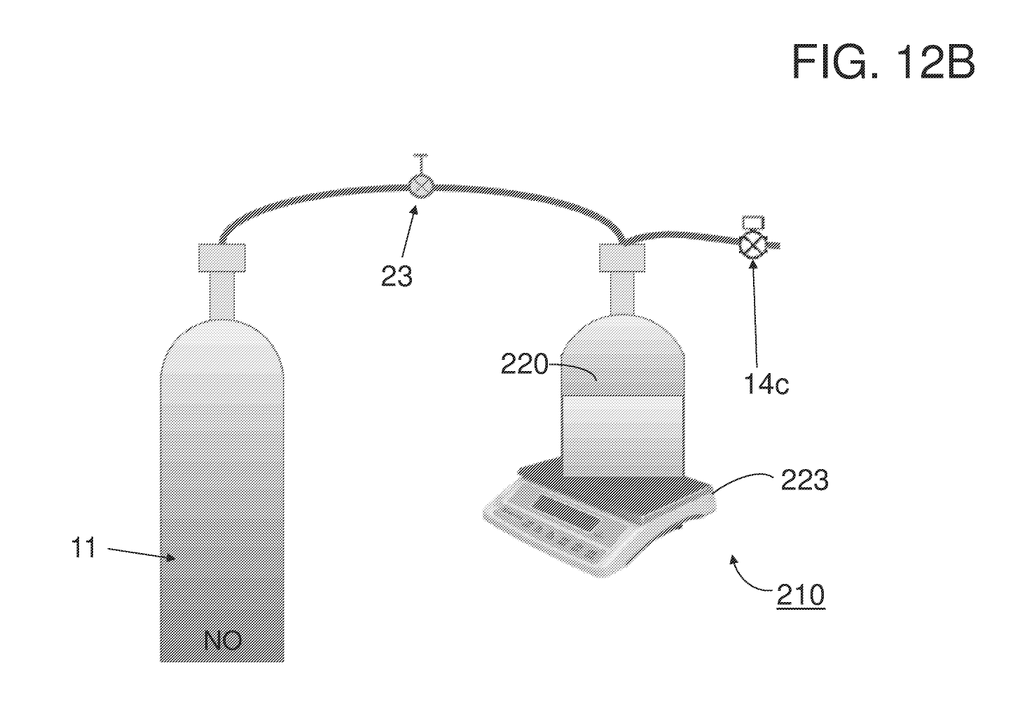

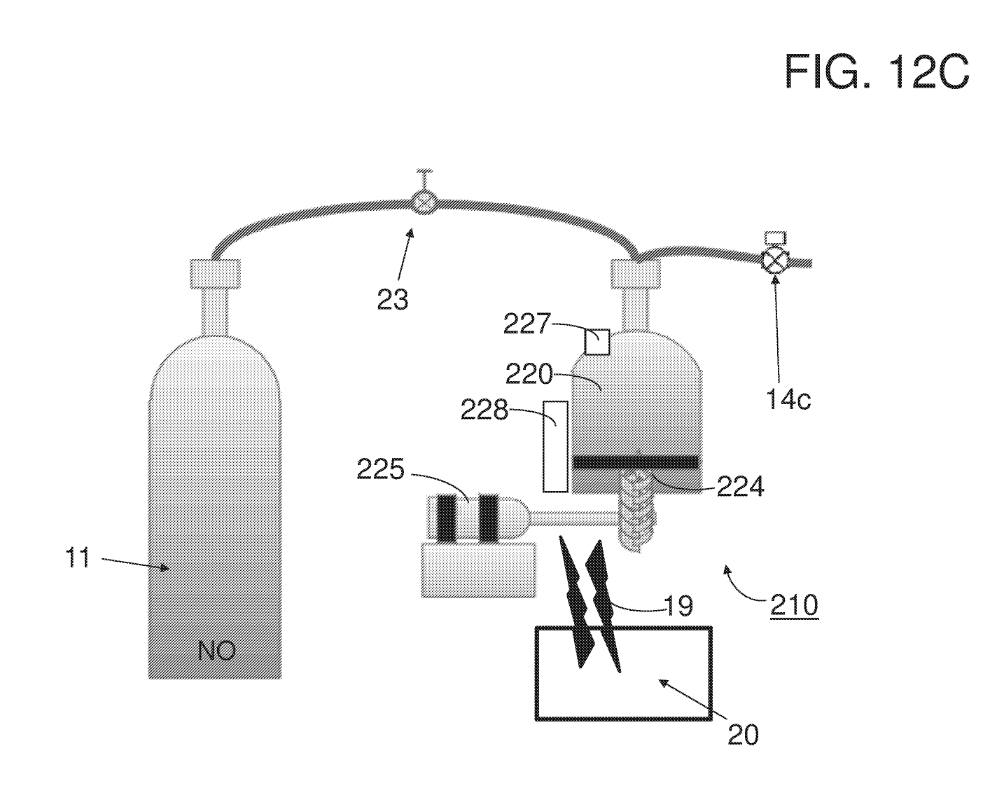

Exemplary embodiments of a GRM system 210, which is in fluid communication with NO container 11 and the valve 14 that controls NO flow into the system according to the present embodiments, are illustrated in FIGS. 12A-C.

GRM system 210 comprises a secondary container that is capable of containing NO at an amount which is smaller than the capacity of NO container 11 but is sufficient to feed the NO inhalation system for at least 2 or at least 3 or at least 4 or at least 5 complete inhalation cycles.

FIG. 12A is a schematic illustration of GRM system 210, which is placed between NO container 11 and valve 14c, and comprises secondary container 220 and buoy 221 that floats on a liquefied content of container 220 and provides an indication of the remaining amount of the liquefied content in secondary container 220 by changing its position. A level meter 222, such as, but not limited to, an electric level meter or an optic level meter transmits data pertaining to the remaining amount of the liquefied content of container 220 to controller 20 (not shown, see, e.g., FIG. 1) via communication link 19.

FIG. 12B is a schematic illustration of GRM system 210, in embodiments in which GRM system 210 is placed between NO container 11 and valve 14c, and comprises mass measuring device 223 which measures the mass of secondary container 220, thereby providing an indication of the remaining amount of NO in secondary container 220. Data pertaining to the remaining amount of the liquefied content of container 220 is transmitted by mass measuring device 223 to controller 20 (not shown, see, e.g., FIG. 1) via communication link 19.

FIG. 12C is a schematic illustration of GRM system 210, in embodiments in which GRM system 210 is placed between NO container 11 and valve 14c, and comprises secondary container 220 which is filled with pressurized gaseous (unliquefied) nitric oxide. The amount of gaseous nitric oxide is optionally and preferably sufficient to feed system 10 for a plurality of cycles as further detailed hereinabove. GRM system 210 can comprise a movable piston 224 within secondary container 220, and a motor 225 configured to displace piston 224, hence to control the pressure in container 220. GRM system 210 can additionally comprise a pressure sensor 227 configured for measuring the gas pressure in container 220. Optionally, but not necessarily GRM system 210 comprises a piston position sensor 228 configured to provide indication regarding the position of piston 224 in container 220. Controller 20 receives via communication link 19 pressure data from pressure sensor 227 and optionally position data from piston position sensor 228, and activates motor 225 so as to maintain a generally constant pressure level in the container. Optionally, the piston position data is used by controller 20 to alert the operator to the amount of NO available in the container or to automatically allow more NO to flow from container 11 to secondary container 220. A person of ordinary skills in the art would know how to determine the required initial amount of NO (“full”) and to calibrate controller 20 to determine any amount between “full” and “empty”.

System Operation Charts:

The system of the present embodiments can be used to produce, deliver and administer any mixture of gases, and particularly gas mixtures of chemical components which can be detected by chemical sensors 17. The system of the present embodiments is particularly but not exclusively, useful to produce, deliver and administer therapeutic mixtures, as defined herein, according to a predetermined administration regimen, as defined herein and/or as provided, for example, in International Patent Application No. PCT/IL2013/050219.

According to some embodiments of the invention, the NO inhalation system is configured to administer intermittent inhalation of the therapeutic mixture in cycles of several minutes, interrupted by periods of several hours during which the subject is allowed to breath ambient air, or the system delivers carrier mixture only and substantially no NO, wherein the fraction of inspired oxygen (FiO₂) in the carrier mixture and in the therapeutic mixture is about 0.21 or higher, and the NO concentration in the therapeutic mixture is about 160 ppm.

The phrase “fraction of inspired oxygen” or “FiO₂”, as used herein, refers to the fraction or percentage of oxygen in a given gas sample. For example, ambient air at sea level includes 20.9% oxygen, which is equivalent to FiO₂ of 0.21. Oxygen-enriched air has a higher FiO₂ than 0.21, up to 1.00, which means 100% oxygen.

The intermittent inhalation regimen may include, according to some embodiments of the present invention, one or more cycles, wherein each cycle is characterized by continuous inhalation of the therapeutic mixture (the gaseous mixture containing NO) at the specified high concentration (e.g., about 140-200 ppm or about 160 ppm) for a first time period, followed by inhalation of a gaseous mixture containing no NO for a second time period. According to some embodiments of the present invention, during the second period of time the subject may inhale ambient air or a controlled mixture of gases which is essentially devoid of NO, referred to herein as an carrier mixture.

In some embodiments, the first time period spans from 10 to 45 minutes, or from 20 to 45 minutes, or from 20 to 40 minutes, and according to some embodiments, spans about 30 minutes.

According to some embodiments of the present invention, the second time period ranges from 3 to 5 hours, or from 3 to 4 hours, and according to some embodiments the second time period spans about 3.5 hours.

According to some embodiments of the present invention, this inhalation regimen is repeated 1-6 times over 24 hours, depending on the duration of the first and second time periods.

In some embodiments, a cycle of intermittent delivery of NO, e.g., 160 ppm for 30 minutes followed by 3.5 hours of breathing no NO, is repeated from 1 to 6 times a day. According to some embodiments, the cycles are repeated 5 times a day.

According to some embodiments of the present invention, the regimen of 1-5 cycles per day is carried out for 1 to 7 days, or from 2 to 7 days, or from 3 to 7 days. According to some embodiments of the present invention, the intermittent inhalation is effected during a time period of 5 days. However, longer time periods of intermittent NO administration using the NO inhalation systems as described herein, are also contemplated.

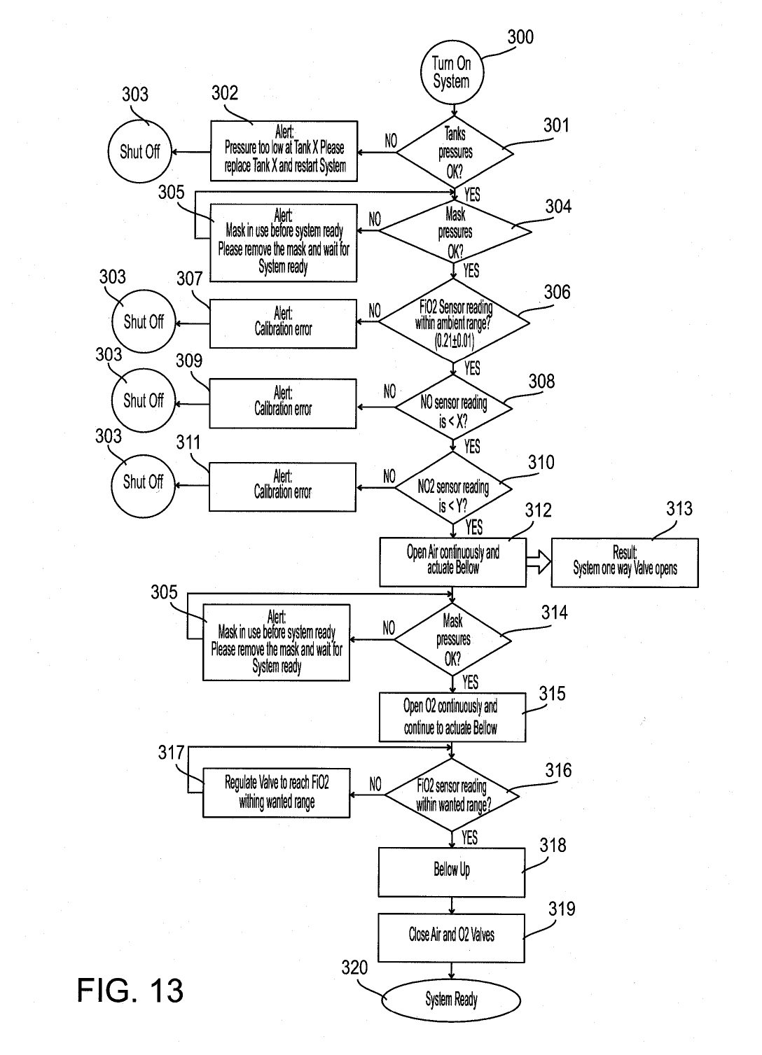

FIG. 13 is a flow chart of a method suitable for initializing an exemplary NO inhalation system according to embodiments of the present invention. The method can be used to check the system and get to a “system ready” state. This method is useful when the system comprises a mask-type inhaler device, an inhaler pressure sensor and actuatable bellows; however, the method is useful when the system comprises other types of inhaler devices.

The method begins at 300 at which the system is turned on, and continues to decision 301 at which the method determines whether the pressure in each of the containers is within a predetermined range. If the pressure in any one of the containers is not within a predetermined threshold range, the method proceeds to 302 at which the method issues an alert signal and then optionally continues to 303 at which the system shuts off.

If the pressures in the containers are within a predetermined threshold range the method proceeds to decision 304 at which the method determines whether the pressure in the mask is within a predetermined range. If the pressure in mask is not within a predetermined threshold range, the method proceeds to 305 at which the method issues an alert signal to remove or discontinue premature use of the mask, and continues to 304 to reexamine the pressure in the mask.

If the pressure in the mask is within a predetermined threshold range the method proceeds to decision 306 at which the method determines whether the FiO₂ chemical sensor is reading the ambient O₂ level within a predetermined range. If the FiO₂ level is not within the expected threshold range, the method proceeds to 307 at which the method issues an alert signal reporting a calibration error pertaining to the FiO₂ chemical sensor, and then optionally continues to 303 at which the system shuts off.

If the FiO₂ level is within a predetermined threshold range the method proceeds to decision 308 at which the method determines whether the NO chemical sensor is reading a zero level within a predetermined range. If the NO level is not within the expected threshold range, the method proceeds to 309 at which the method issues an alert signal reporting a calibration error pertaining to the NO chemical sensor, and then optionally continues to 303 at which the system shuts off.

If the NO level is within a predetermined threshold range the method proceeds to decision 310 at which the method determines whether the NO₂ chemical sensor is reading a zero level within a predetermined range. If the NO₂ level is not within the expected threshold range, the method proceeds to 311 at which the method issues an alert signal reporting a calibration error pertaining to the NO₂ chemical sensor, and then optionally continues to 303 at which the system shuts off.

If the NO₂ level is within a predetermined threshold range the method proceeds to 312 at which the method sends a signal to open the one-way electric valve in the system to allow air (ambient oxygen levels and no nitric oxide) to flow continuously, actuates the bellows and proceeds to decision 314 at which the method determines whether the pressure in the mask is within a predetermined range. If the pressure in mask is not within a predetermined threshold range, the method proceeds to 305 at which the method issues an alert signal to remove or discontinue premature use of the mask, and continues to 314 to reexamine the pressure in the mask.

If the pressure in the mask is within a predetermined threshold range the method proceeds to 315 at which the method increases the oxygen level to a predetermined level and proceeds to decision 316 at which the method checks if the FiO₂ level in the system is within the predetermined elevated range. If the FiO₂ level is not within a predetermined threshold range, the method proceeds to 317 at which the method adjusts FiO₂ by returning to 316.

If the FiO₂ level is within a predetermined elevated threshold range the method proceeds to 318 at which the method sends a signal to raise the bellows which is then filled with a carrier mixture at a predetermined FiO₂ level, the method then proceeds to 319 at which the air and O₂ valves are closed, at which point the method proceeds to 320 “system ready” and the system is ready to be used.

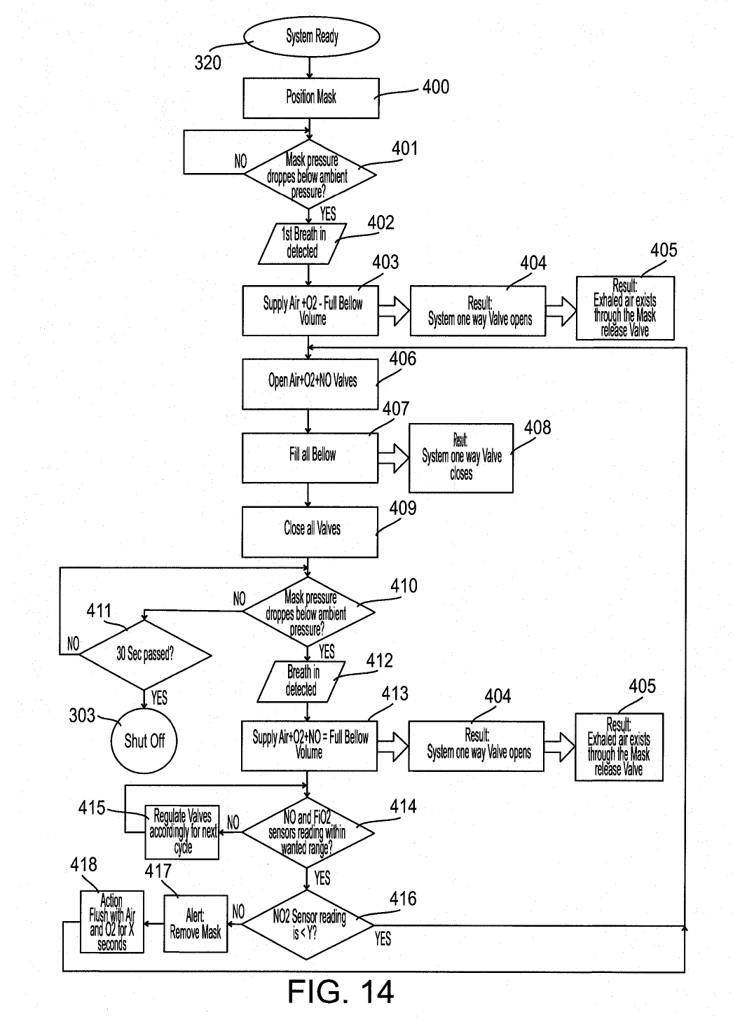

FIG. 14 is a flow chart of a method suitable for operating an exemplary NO inhalation system according to embodiments of the present invention. The method is useful when the system comprises a mask-type inhaler device, an inhaler pressure sensor and actuatable bellows, to deliver a therapeutic mixture to a subject; however, the method is useful when the system comprises other types of inhaler devices.

The method begins at 320 at which the system is ready for use, after running the method described above in connection with FIG. 13. The method proceeds to 400 at which the method issues a signal to position the mask on the subject, and thereafter the method proceeds to decision 401 at which the method determines whether the pressure in the mask is below ambient pressure within a predetermined range. If the pressure in mask is not within a predetermined threshold range, the method repeats 401 until the result is within a predetermined threshold range. Once the pressure in the mask indicates a low pressure, the method proceeds to 402 at which the method detects a breath via the mask's pressure sensor, and issues a signal to actuate the bellows and supply its contents of carrier mixture (403), the one-way valve opens (404) and exhalation exits through the mask's outlets and valves (405).

The method then proceeds to 406 at which the method opens the air or N₂ valve, the O₂ valve and the NO valve and proceeds to 407 at which the bellows is filled with a therapeutic gas mixture, and to 408 at which the one-way valve opens. The method then proceeds to 409 at which all valves are closed and to decision 410 at which the method determines whether the pressure in the mask is below ambient pressure within a predetermined range.

If at 410 the pressure in mask is not within a predetermined threshold range, the method proceeds to decision 411 at which the method holds for a short period of time (5-60 seconds, or 30 seconds for example), during which the pressure in the mask is monitored. If within the short period of time the pressure in the mask is not within a predetermined threshold range that indicates that a breath is being taken by the subject, the methods proceeds to 303 and stops. If a breath is detected (412), the method actuates the bellows (413) and supplies its contents (e.g., a therapeutic mixture), the one-way valve opens (404) and exhalation exits through the mask's outlets and valves (405).

The method then proceeds to decision 414 at which the method monitors the chemical sensors of FiO₂ and NO to determine if the mixture is within the predetermined threshold range. If the levels of FiO₂ and NO are not within the predetermined threshold range, the method proceeds to 415 at which the valves of the source containers are regulated to achieve the desired mixture.

If the mixture is detected as the desired mixture, the method proceeds to 416 at which the method determined the NO₂ level is within predetermined threshold range. If the NO₂ level is within predetermined threshold range, the method proceeds to 406 and the cycle repeats. If the NO₂ level exceeds the predetermined threshold range, the method proceeds to 417 at which the method issues an alert signal to remove the mask and proceeds to 418 at which the method actuates the actuatable flushing valve to rid the mask of its contents, and the method proceeds to 406 and the cycle repeats.

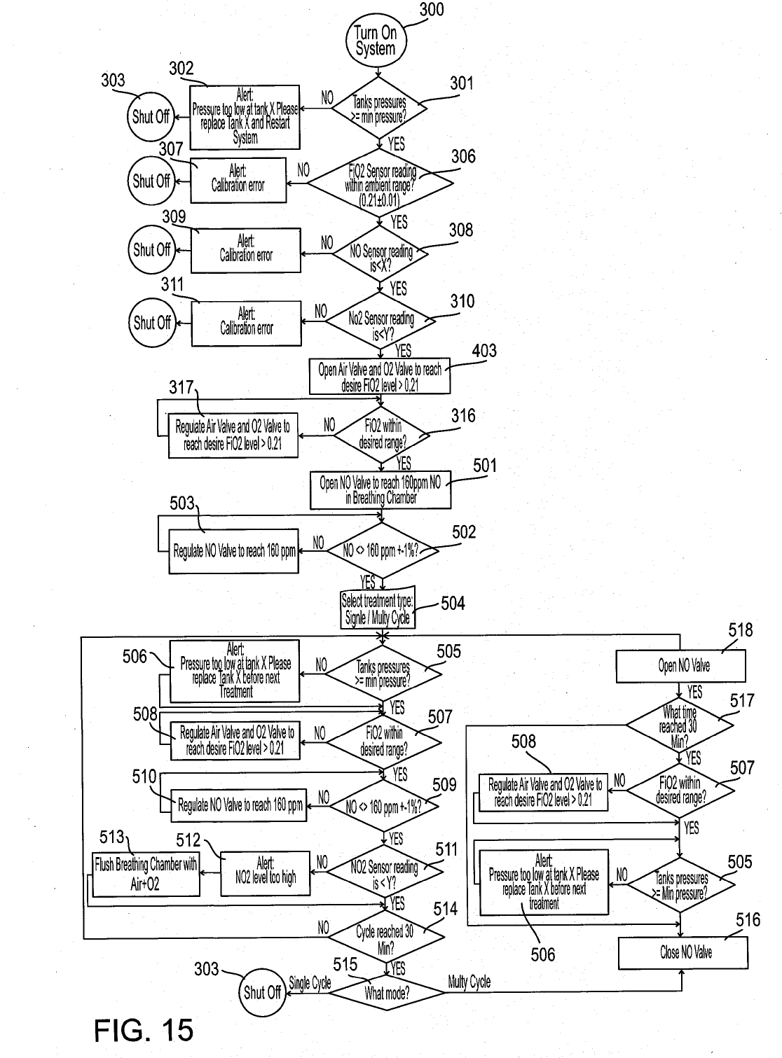

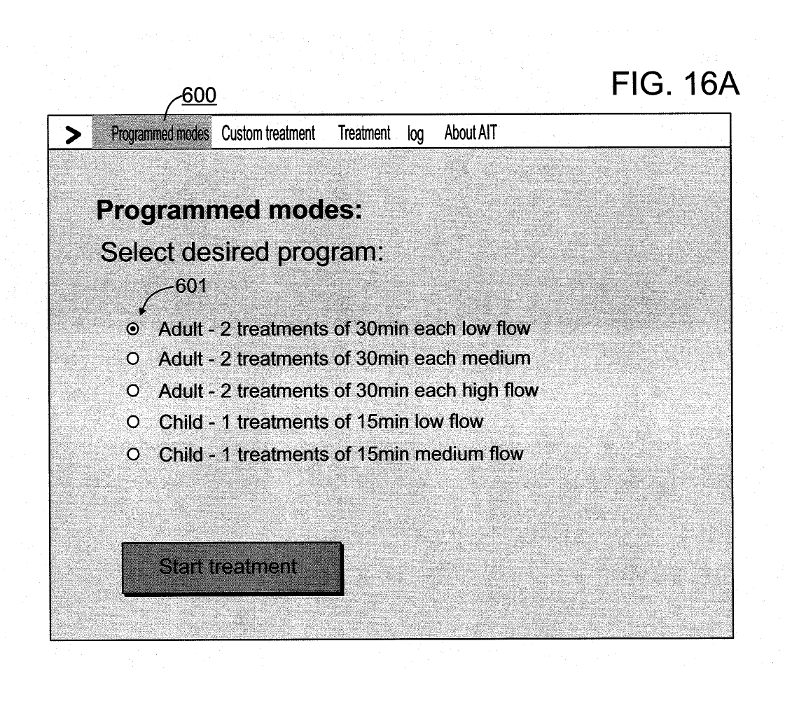

FIG. 15 is a flow chart of a method suitable for operating an exemplary NO inhalation system according to embodiments of the present invention. The method is particularly useful when the system comprises a mask-type inhaler device, an inhaler pressure sensor and actuatable bellows; however, the method is useful when the system comprises other types of inhaler devices. The method combines the initialization method described above in connection with FIG. 13 and the delivery method described above in connection with FIG. 14.

As shown in FIG. 15, once the system is initialized and the chemical composition of the flow is within predetermined threshold parameters in terms of FiO₂, the method proceeds to 501 at which the NO valve is opened.

The method proceeds to decision 502 at which the data from the NO chemical sensor is received and the method proceeds to 503 at which the data processor analyses the data and indicates that the level of NO has reached at least 160 ppm, thereby signaling that the therapeutic mixture is ready.

When the therapeutic mixture is ready the method proceeds to 504 at which the GUI is used to select the type of treatment, e.g., a single cycle of multiple cycles, and the method proceeds to self-check (505 and 506), maintain FiO₂ level (507 and 508), maintain the NO concentration in the therapeutic mixture (509 and 510) while monitoring NO₂ levels (511) and responding by alerting and flushing the inhaler device accordingly (512 and 513).

The method then proceeds to 514 at which the method repeats 505-513 for a predetermined time period that constitutes a single cycle of treatment.

When the cycle is completed, the method proceeds to 515 at which the method either stops (303) in case of a single cycle mode, or proceeds to 516 at which the NO valve is closed, while the system maintains self-check (505 and 506) and FiO₂ level (507 and 508) and the method proceeds to 517 at which the method repeats 505-508 for predetermined time period.

When the predetermined time period at 517 has passed, the method proceeds to 518 at which the NO valve opens and the method proceeds to repeat 505-515.

Graphical User Interface:

The NO inhalation system presented herein can be operated by means of a graphical user interface (GUI), which includes the controller of the system, according to some embodiments of the present invention. The GUI allows the operator of the system to interact with various electronic elements of the system through graphical icons and visual indicators such as secondary notation, as opposed to text-based interfaces, typed command labels or text navigation. The actions in the GUI are performed through direct manipulation of the graphical elements therein which are presented on a screen. The screen can form a part of a desktop display apparatus or a hand-held display apparatus. The graphical elements of the GUI can be manipulated by physical input devices (mouse, buttons and switches) or by touching the elements in a touch-sensitive/responsive display apparatus.

According to embodiments of the present invention, the graphical user interface (GUI) is designed for an inhalation system as presented herein, is having a controller configured for delivering to an inhaler device a therapeutic mixture which comprises NO, and for controlling a flow of the therapeutic mixture responsively to a concentration of at least NO, O₂ and NO₂ in the therapeutic mixture. According to some embodiments, the GUI includes at least one of:

a first display area for displaying a plurality of treatment protocols and allowing a user to select one treatment protocol;

a second display area for displaying controller interface configured to communicate user selection data to the controller;

a third display area displaying data pertaining to the concentration of NO, O₂ and NO₂ in the therapeutic mixture during the delivery of the therapeutic mixture; and

a fourth display area for displaying treatment log data.

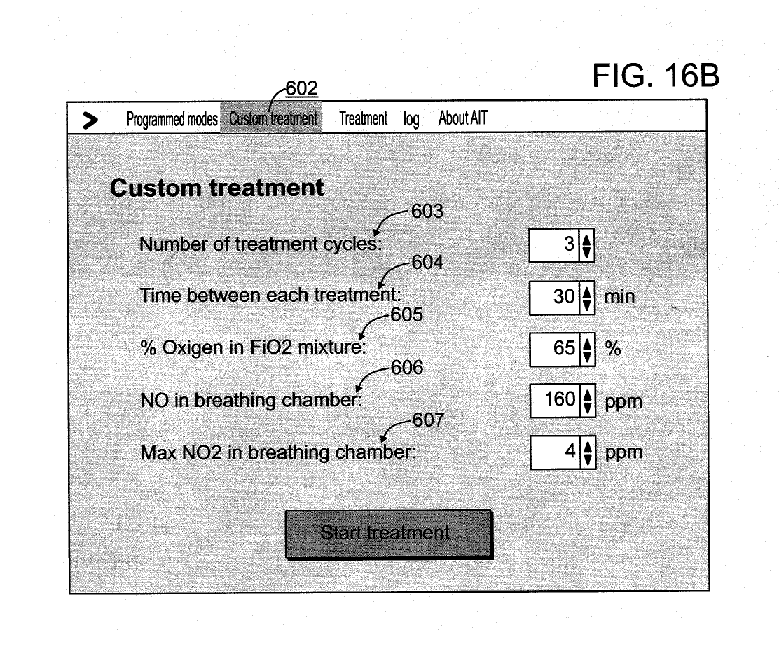

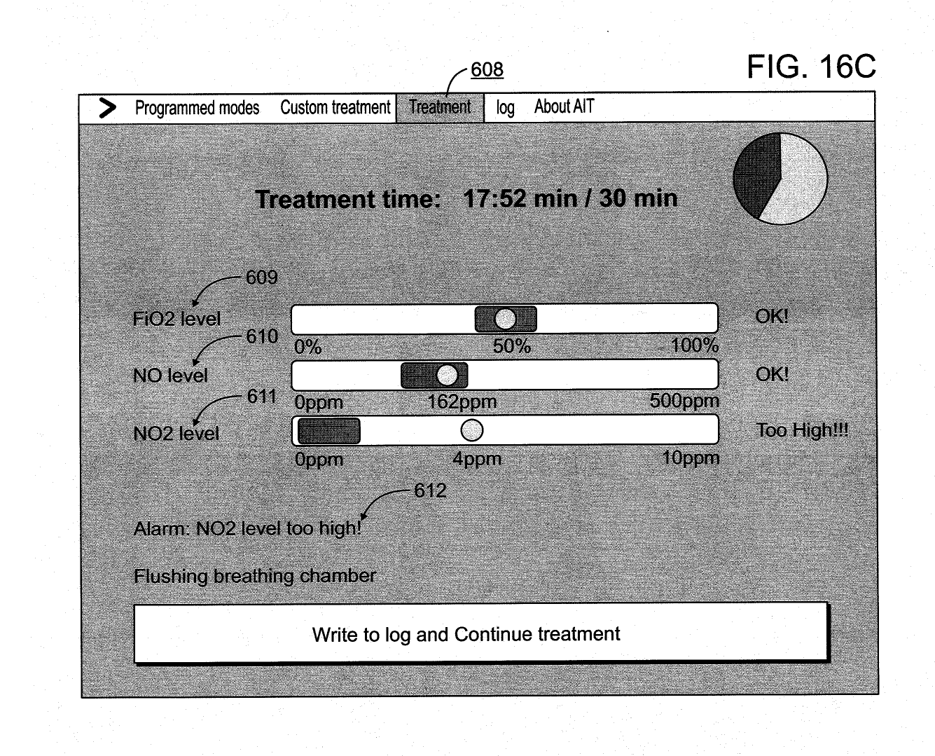

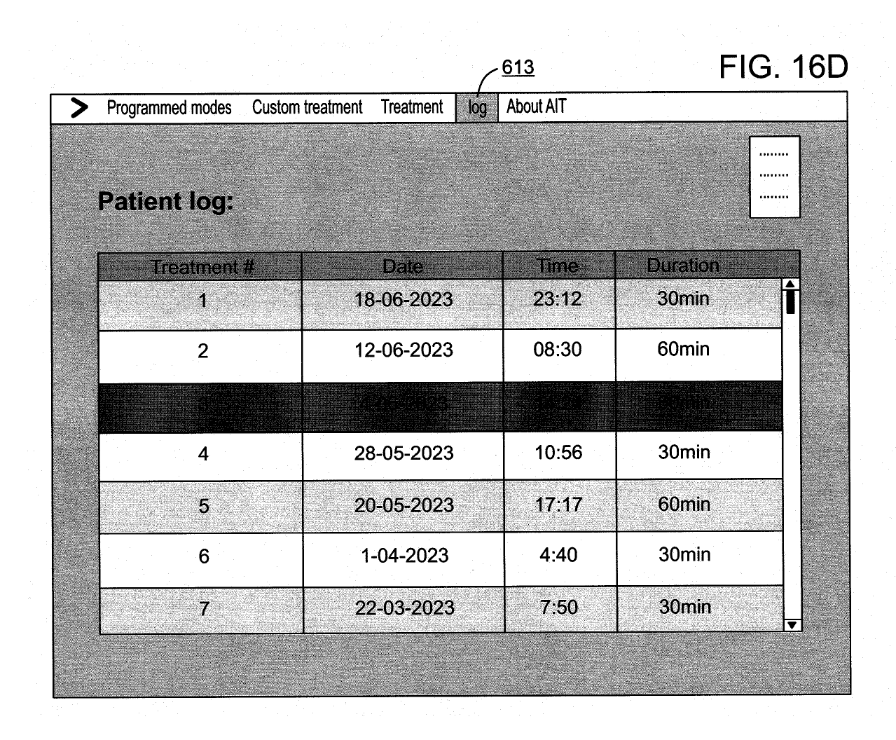

FIGS. 16A-D present exemplary graphical elements of an exemplary GUI according to embodiments of the present invention, suitable for operating the NO inhalation system presented herein. FIG. 16A and FIG. 16B present exemplary treatment program selection operations, referred to as a “first display area” 600. In FIG. 16A, the GUI displays a plurality of treatment protocols 601 and allow a user to select one treatment protocol. In FIG. 16B the GUI display a plurality of adjustable parameters, referred to as a “second display area” 602 (e.g., number of treatment cycles (603), time period between cycles (604), desired FiO₂ (605), desired NO level in the inhaler device (606) and NO2 threshold level (607)) allowing the user to set the values of one or more parameters to be used in the treatment. GUI comprises a controller interface (not shown) configured to communicate the user selection data (e.g., the selected protocol of FIG. 16A and/or the parameters of FIG. 16B). FIG. 16C presents an exemplary “third display area” 608 displaying data pertaining to the concentration of NO 609, O₂ 610 and NO₂ 611 in the therapeutic mixture during the delivery of the therapeutic mixture, which is useful in monitoring stage which is displayed during operation of the system. In the example presented in FIG. 16C the NO₂ levels are exceeded, displaying alert 612 the operator that the system is actuating the actuatable flushing valve. FIG. 16D presents an exemplary patient treatment logs which can form a fourth display area 613 for recalling treatment data to be repeated or changed according to the operator's discretion.

It is expected that during the life of a patent maturing from this application many relevant NO inhalation systems will be developed and the scope of the term NO inhalation system is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A system for inhalation, comprising:
gas supply apparatus configured to separately supply at least NO, and a carrier gas mixture which contains O₂;
a mixer apparatus configured for receiving gases from said supply apparatus and mixing said NO with said carrier gas mixture to provide a therapeutic mixture;
an inhaler device configured for receiving said therapeutic mixture and releasing said therapeutic mixture in an enclosed space of said inhaler device;
a chemical sensing assembly configured for providing data pertaining to a concentration of at least NO₂ in said inhaler device; and
a controller configured for controlling flow of said therapeutic mixture responsively to said data.

2. The system of claim 1, wherein said chemical sensing assembly is further configured for providing data pertaining to a concentration of each of NO and O₂ independently in said inhaler device.

3. The system of claim 1, wherein said mixer apparatus comprises:
a mixing chamber formed with a first inlet port for receiving NO gas, and a second inlet port for receiving an additional gas; and
a rotatable member mounted in said mixing chamber and configured for rotating within said mixing chamber so as to mix said NO with said additional gas.

4. The system of claim 1, wherein said controller comprises:
a data processor configured for receiving said data and calculating flow parameters responsively to said data, the controller is configured for controlling flow of said therapeutic mixture based on said calculated flow parameters.

5. The system of claim 1, further comprising a graphical user interface (GUI).

6. The system of claim 5, wherein said GUI comprises:
a first display area for displaying a plurality of treatment protocols and allowing a user to select one treatment protocol;
a second display area for displaying controller interface configured to communicate user selection data to said controller;
a third display area for displaying said data during delivery of said therapeutic mixture; and
a fourth display area for displaying treatment log data.

7. The system of claim 1, wherein said gas supply apparatus comprises a gas reservoir monitoring system.

8. The system of claim 7, wherein said gas reservoir monitoring system comprises:
a container capable of containing a predetermined amount of pressurized nitric oxide (NO);
a movable piston in said container; and
a pressure sensor,
said controller being configured to adjust a position of said piston responsively to pressure data received from said pressure sensor so as to maintain a generally constant pressure level in said container.

9. The system of claim 8, wherein said gas reservoir monitoring system further comprises a piston position sensor,
said controller being configured to received position data from said piston position sensor, and analyze and display said position data.

10. The system of claim 8, wherein said gas reservoir monitoring system is connectable to said gas supply apparatus.

11.-25. (canceled)

26. A mixer apparatus for an inhalation system, the mixer apparatus comprising:
a mixing chamber formed with a first inlet port for receiving NO gas, and a second inlet port for receiving an additional gas; and
a rotatable member mounted in said mixing chamber and configured for rotating within said mixing chamber so as to mix said NO with said additional gas to provide a flow of a therapeutic mixture.

27.-28. (canceled)

29. A controller system for an inhalation system, comprising:
a data processor, configured for receiving data pertaining to concentration of each of NO, O₂ and NO₂ independently and calculating flow parameters responsively to said data; and
a controller configured for controlling flow of a therapeutic mixture which comprises NO in the inhalation system based on said calculated flow parameters.

30. The controller system of claim 29, wherein said controller is configured for controlling flow of NO responsively to said data pertaining to concentration of NO so as to reach an NO concentration of at least 160 ppm.

31. The controller system of claim 29, wherein said controller is configured for actuating an actuatable flushing valve responsively to said data pertaining to concentration of NO₂.

32. A system for inhalation, comprising:
a head respiratory hood adapted to be worn over the head of a subject and having an inlet port and an outlet port; and
a supply and control system configured to introduce into said inlet port a therapeutic mixture which comprises NO, to provide data pertaining to a concentration of at least NO₂ in said therapeutic mixture, and to control a flow of said therapeutic mixture responsively to said data.

33. A system for inhalation, comprising:
a whole body respiratory encapsulation adapted to encapsulate the entire body of a subject and having an inlet port and an outlet port; and
a supply and control system configured to introduce into said inlet port a therapeutic mixture which comprises NO, to provide data pertaining to a concentration of at least NO₂ in said therapeutic mixture, and to control a flow of said therapeutic mixture responsively to said data.

34. A system for inhalation, comprising:
a generally closed enclosure adapted to contain a plurality of mammalian subjects and having an inlet port and an outlet port; and
a supply and controller system configured to introduce into said inlet port a therapeutic mixture which comprises NO, to provide data pertaining to a concentration of at least NO₂ in said therapeutic mixture, and to control a flow of said therapeutic mixture responsively to said data.

35. The system, apparatus or interface of any one of claims 1-34, wherein the system further comprises an actuatable valve configured responsively to said data pertaining to a concentration of NO₂.

36.-100. (canceled)
